(19) United States
(12) Patent Application Publication
(10) Pub. No.: US 2026/0150493 A1
(43) Pub. Date: May 28, 2026

(54) COMPOUND AND POLYMER AND ELECTROCHROMIC DEVICE AND ELECTRONIC DEVICE

(71) Applicant: Samsung Electronics Co., Ltd., Suwon-si (KR)

(72) Inventors: Tae Jin CHOI, Suwon-si (KR); Jeoung In YI, Suwon-si (KR); Youngnam KWON, Suwon-si (KR); Jun Cheol BAE, Suwon-si (KR); Heejae LEE, Suwon-si (KR); Won-Jae JOO, Suwon-si (KR)

(73) Assignee: Samsung Electronics Co., Ltd., Suwon-si (KR)

(21) Appl. No.: 19/386,921

(22) Filed: Nov. 12, 2025

(30) Foreign Application Priority Data
Nov. 26, 2024 (KR) 10-2024-0171407

(51) Int. Cl. H10K 50/13; C07D 497/04; H10K 50/86; H10K 59/38; H10K 59/50; H10K 59/80; H10K 85/10; H10K 85/60

(52) U.S. Cl. CPC H10K 50/131; C07D 497/04; H10K 50/865; H10K 59/38; H10K 59/50; H10K 59/8792; H10K 85/1135; H10K 85/626; H10K 85/636

(57) ABSTRACT

Disclosed are a compound represented by Chemical Formula 1, a polymer including a structural unit derived from the compound, and an electrochromic device, and an electronic device including the same.

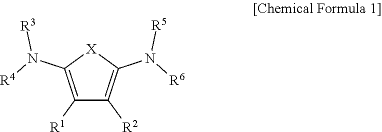

In Chemical Formula 1, X and R¹ to R⁶ are the same as defined in the specification.

### CROSS-REFERENCE TO RELATED APPLICATION

This application claims priority to and the benefit of Korean Patent Application No. 10-2024-0171407 filed with the Korean Intellectual Property Office on Nov. 26, 2024, the entire contents of which are incorporated herein by reference.

### BACKGROUND

### 1. Field

The present inventive concepts relate to compounds, polymers, electrochromic devices, and electronic devices.

### 2. Description of the Related Art

Electrochromic devices may control inflow of light by controlling the color of electrochromic materials through electrochemical redox reactions, and thus may be applied to various fields such as optical filters, electronic signboards, smart windows, or wearable imaging devices.

### SUMMARY

Some example embodiments provide a compound that may be used as an electrochromic material to achieve effective color display control.

Some example embodiments provide a polymer of the compound that may be used as an electrochromic material to achieve effective color display control.

Some example embodiments provide an electrochromic device including the compound and/or the polymer.

Some example embodiments provide a stacked electrochromic device including the compound and/or the polymer.

Some example embodiments provide an electronic device including the compound, the polymer, the electrochromic device, and/or the stacked electrochromic device.

According to some example embodiments, a compound represented by Chemical Formula 1 may be provided.

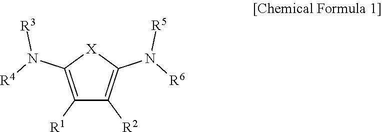

In Chemical Formula 1,
X may be O, S, Se, or Te,
R¹ and R² may each independently be hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
at least one of R¹ or R² may be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
R¹ and R² may each independently be present or may be linked to each other to form a ring,
R³ to R⁶ may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
at least one of R³ or R⁴ may be a substituted or unsubstituted C6 to C30 aryl group, and
at least one of R⁵ or R⁶ may be a substituted or unsubstituted C6 to C30 aryl group.

At least one of R³ or R⁴ may be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group and at least one of R⁵ or R⁶ may be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group.

R¹ and R² may each independently be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, or any combination thereof, and R¹ and R² may each independently be present or linked to each other to form a ring.

The compound may be represented by Chemical Formula 1A.

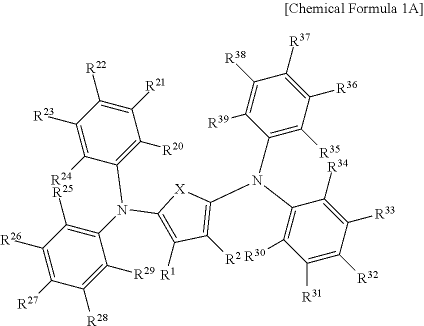

In Chemical Formula 1A,
X may be O, S, Se, or Te,
R¹ and R² may each independently be hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
at least one of R¹ or R² may be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
R¹ and R² may each independently be present or linked to each other to form a ring,
R²⁰ to R³⁹ may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, a hydroxyl group, a halogen, or any combination thereof, and
R²⁰ to R³⁹ may each independently be present or two adjacent ones of R²⁰ to R³⁹ may be linked to each other to form a ring.

The compound may be represented by Chemical Formula 1B-1 or Chemical Formula 1B-2.

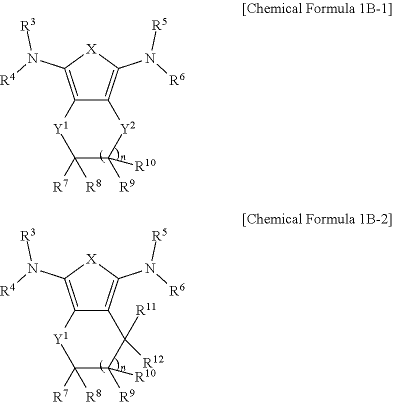

In Chemical Formula 1B-1 and Chemical Formula 1B-2,
X, Y¹, and Y² may each independently be O, S, Se, or Te,
R³ to R¹² may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
R⁷ to R¹² may each independently be present or two adjacent ones of R⁷ to R¹² may be linked to each other to form a ring,
at least one of R³ or R⁴ may be a substituted or unsubstituted C6 to C30 aryl group,
at least one of R⁵ or R⁶ may be a substituted or unsubstituted C6 to C30 aryl group, and
n may be an integer of 0 to 3.

The compound may be represented by Chemical Formula 1C-1 or Chemical Formula 1C-2.

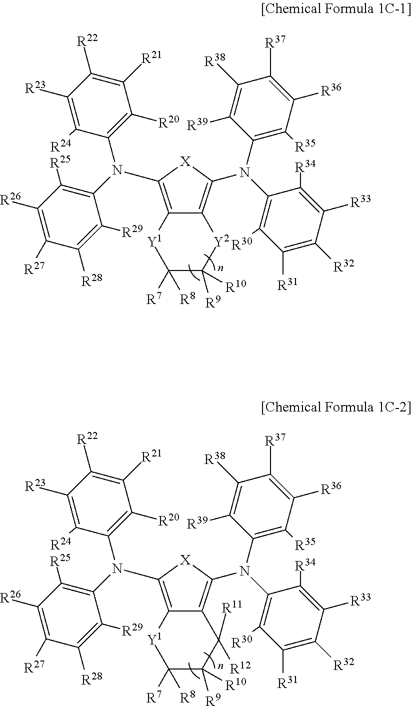

In Chemical Formula 1C-1 and Chemical Formula 1C-2,
X, Y¹, and Y² may each independently be O, S, Se, or Te,
R⁷ to R¹² and R²⁰ to R³⁹ may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
R⁷ to R¹² may each independently be present or two adjacent ones of R⁷ to R¹² may be linked to each other to form a ring,
R²⁰ to R³⁹ may each independently be present or two adjacent ones of R²⁰ to R³⁹ may be linked to each other to form a ring, and
n may be an integer of 0 to 3.

The compound may be an electrochromic material that is configured to be reversibly converted, based on an applied voltage into a colored state wherein the electrochromic material is configured to absorb at least a portion of light in a visible light region to display a color, or a bleached state wherein the electrochromic material is configured to transmit light in the visible light region, the transmitted light including the portion of light in the visible light region.

According to some example embodiments, a polymer including a structural unit derived from the compound may be provided.

According to some example embodiments, an electrochromic device may include a first electrode and a second electrode, a first electrochromic layer between the first electrode and the second electrode and including a first electrochromic material, and a first electrolyte in contact with the first electrochromic layer, wherein the first electrochromic material may include the compound, a polymer including a structural unit derived from the compound, or any combination thereof.

The polymer may be a reaction product of electro-polymerization using at least one of R³ to R⁶ of Chemical Formula 1 as a reaction site.

The electrochromic device may be configured to exhibit a difference in light transmittance at a wavelength of 470 nm based on a difference in an applied voltage that is applied to the electrochromic device, where the difference in light transmittance may be about 30% to about 100%.

The electrochromic device may further include a second electrochromic layer between the first electrode and the second electrode and including a second electrochromic material different from the first electrochromic material, and the second electrochromic material may be configured to be reversibly converted into a colored state wherein the second electrochromic material is configured to absorb at least a portion of light in a visible light region to display a color or a bleached state wherein the second electrochromic material is configured to transmit light in the visible light region, the transmitted light including the portion of light in the visible light region.

The first electrochromic material may be configured to exhibit a first maximum absorption wavelength in the visible light region based on being reversibly converted to a colored state of the first electrochromic material to be configured to absorb at least a portion of light in the visible light region. The second electrochromic material may be configured to exhibit a second maximum absorption wavelength in the visible light region based on being reversibly converted to the colored state of the second electrochromic material. The second maximum absorption wavelength of the second electrochromic material may be different from the first maximum absorption wavelength of the first electrochromic material.

The electrochromic device may be configured to reversibly display black or transparency based on the applied voltage, and the electrochromic device may be configured to exhibit a difference in light transmittance in the visible light region based on a difference in the applied voltage, wherein the difference in light transmittance in the visible light region is about 30% to about 100%.

According to some example embodiments, a stacked electrochromic device may include a first electrochromic device, which may include the electrochromic device, and a second electrochromic device stacked with the first electrochromic device, wherein the second electrochromic device may include a third electrode and a fourth electrode, a second electrochromic layer between the third electrode and the fourth electrode, and a second electrolyte in contact with the second electrochromic layer.

The second electrochromic layer may include a second electrochromic material that is different from the first electrochromic material and is configured to, based on an applied voltage, be reversibly converted into a colored state wherein the second electrochromic material is configured to absorb at least a portion of light in a visible light region to display a color, or a bleached state wherein the second electrochromic material is configured to transmit light in the visible light region, the transmitted light including the portion of light in the visible light region.

The first electrochromic material may be configured to exhibit a first maximum absorption wavelength in the visible light region based on being reversibly converted to a colored state of the first electrochromic material to be configured to absorb at least a portion of light in the visible light region. The second electrochromic material may be configured to exhibit a second maximum absorption wavelength in the visible light region based on being reversibly converted to the colored state of the second electrochromic material. The second maximum absorption wavelength of the second electrochromic material may be different from the first maximum absorption wavelength of the first electrochromic material.

The stacked electrochromic device may be configured to reversibly display black or transparency based on an applied voltage, and the electrochromic device may be configured to exhibit a difference in light transmittance in the visible light region based on a difference in the applied voltage, wherein the difference in light transmittance in the visible light region is about 30% to about 100%.

According to some example embodiments, an electronic device including the electrochromic device or the stacked electrochromic device is provided.

The electrochromic material may be capable of effectively controlling electrochemical and optical properties thereby realizing improved electrochromic properties, including more effective color display control.

### BRIEF DESCRIPTION OF THE DRAWINGS

FIG. 1 is a cross-sectional view illustrating an electrochromic device according to some example embodiments,

FIG. 2 is a cross-sectional view illustrating an electrochromic device according to some example embodiments,

FIG. 3 is a cross-sectional view illustrating an electrochromic device according to some example embodiments,

FIG. 4 is a schematic view of an electronic device according to some example embodiments,

FIG. 5 is a UV-Vis absorption spectrum of the electrochromic device according to Example 1 in a colored state and a bleached state,

FIG. 6 is a UV-Vis absorption spectrum of an electrochromic device according to Example 2 in a colored state and a bleached state,

FIG. 7 is a UV-Vis absorption spectrum of the electrochromic device according to Comparative Example 1 in a colored state and a bleached state,

FIG. 8 is a UV-Vis absorption spectrum of the electrochromic device according to Comparative Example 2 in a colored state and a bleached state,

FIG. 9 is a UV-Vis absorption spectrum of the electrochromic device according to Example 3 in a colored state and a bleached state, and

FIG. 10 shows the transmission spectra of the electrochromic device according to Example 4 in the colored and bleached states.

### DETAILED DESCRIPTION

Hereinafter, some example embodiments of the present inventive concepts will be described in detail so that a person skilled in the art would understand the same. However, the inventive concepts may be embodied in many different forms and are not to be construed as limited to the example embodiments set forth herein.

In the drawings, the thickness of layers, films, panels, regions, etc., are exaggerated for clarity. It will be understood that when an element such as a layer, film, region, or substrate is referred to as being “on” another element, it may be directly on the other element or intervening elements may also be present. In contrast, when an element is referred to as being “directly on” another element, there are no intervening elements present. When an element is described as being “on” another element, the element may be above, beneath, or horizontally adjacent to the other element.

In the drawings, parts having no relationship with the description are omitted for clarity, and the same or similar constituent elements are indicated by the same reference numeral throughout the specification.

Hereinafter, the terms “lower portion” and “upper portion” are for convenience of description and do not limit the positional relationship.

Hereinafter, the term “combination” includes mixed and two or more laminated or stacked structures.

It will be understood that elements and/or properties thereof may be recited herein as being “identical”, “the same”, or “equal” as other elements and/or properties thereof, and it will be further understood that elements and/or properties thereof recited herein as being “identical” to, “the same” as, or “equal” to other elements and/or properties thereof may be “identical” to, “the same” as, or “equal” to or “substantially identical” to, “substantially the same” as or “substantially equal” to the other elements and/or properties thereof. Elements and/or properties thereof that are “substantially identical” to, “substantially the same” as or “substantially equal” to other elements and/or properties thereof will be understood to include elements and/or properties thereof that are identical to, the same as, or equal to the other elements and/or properties thereof within manufacturing tolerances and/or material tolerances. Elements and/or properties thereof that are identical or substantially identical to, equal to or substantially equal to, and/or the same or substantially the same as other elements and/or properties thereof may be structurally the same or substantially the same, functionally the same or substantially the same, and/or compositionally the same or substantially the same. While the term “same,” “equal” or “identical” may be used in description of some example embodiments, it should be understood that some imprecisions may exist. Thus, when one element or property is referred to as being identical to, equal to, or the same as another element or property, it should be understood that the element or property is the same as another element or property within a desired manufacturing or operational tolerance range (e.g., ±10%).

It will be understood that elements and/or properties thereof described herein as being “substantially” the same, equal, and/or identical encompasses elements and/or properties thereof that have a relative difference in magnitude that is equal to or less than 10%. Further, regardless of whether elements and/or properties thereof are modified as “substantially,” it will be understood that these elements and/or properties thereof should be construed as including a manufacturing or operational tolerance (e.g., ±10%) around the stated elements and/or properties thereof.

When the terms “about” or “substantially” are used in this specification in connection with a numerical value, it is intended that the associated numerical value includes a manufacturing or operational tolerance (e.g., ±10%) around the stated numerical value. Moreover, when the words “about” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes. When ranges are specified, the range includes all values therebetween such as increments of 0.1%.

As described herein, when an operation is described to be performed, or an effect such as a structure is described to be established “by” or “through” performing additional operations, it will be understood that the operation may be performed and/or the effect/structure may be established “based on” the additional operations, which may include performing said additional operations alone or in combination with other further additional operations.

As used herein, when a definition is not otherwise provided, “substituted” refers to replacement of a hydrogen atom of a compound or a group by a substituent selected from a halogen, a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a C1 to C30 alkyl group, a C2 to C30 alkenyl group, a C2 to C30 alkynyl group, a C6 to C30 aryl group, a C3 to C30 heteroaryl group, a C7 to C30 arylalkyl group, a C1 to C30 alkoxy group, a C1 to C20 heteroalkyl group, a C3 to C20 heterocyclic group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and any combination thereof. When a group has two or more substituents, the substituents may each be present independently, or two adjacent substituents may be linked to each other to form a ring.

As used herein, when a definition is not otherwise provided, “hetero” refers to one including 1 to 4 heteroatoms selected from N, O, S, Se, Te, Si, and P.

Hereinafter, “polymer” includes a homopolymer, a copolymer, or any combination thereof.

Hereinafter, the term “combination” includes a mixture, composite, or stacked structure of two or more.

Hereinafter, a compound according to some example embodiments is described.

A compound according to some example embodiments is represented by Chemical Formula 1.

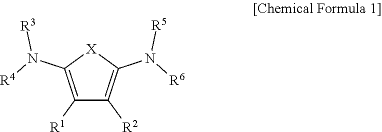

In Chemical Formula 1,
X may be O, S, Se, or Te,
R¹ and R² may each independently be hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
at least one of R¹ or R² may be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
R¹ and R² may each independently be present or linked to each other to form a ring,
R³ to R⁶ may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
at least one of R³ or R⁴ may be a substituted or unsubstituted C6 to C30 aryl group, and
at least one of R⁵ or R⁶ may be a substituted or unsubstituted C6 to C30 aryl group.

The compound represented by Chemical Formula 1 may have a plurality of substituents capable of controlling electrochemical oxidation and reduction reactions at a core of a pentagonal heterocycle capable of controlling electrical properties or a fused ring thereof. By combining these core and substituents, the compound may be configured to be reversibly converted to (e.g., reversibly converted between) a state (opaque, colored state) wherein the compound is configured to absorb at least a portion of light in the visible light region, including for example light in one or more limited wavelength bands or wavelength regions within the visible light region, and/or a state (transparent, bleached state) wherein the compound is configured not to absorb light in the visible light region including not absorbing the portion of light absorbed in the colored state, for example to transmit light in the visible light region (e.g., light in an entirety or substantially an entirety of the visible light region), including transmitting the portion of light that is absorbed when the compound is in the colored state, at a relatively fast rate through electrochemical oxidation and reduction according to the applied voltage (e.g., a voltage applied to the compound), so that the compound may be effectively applied (e.g., used) as an electrochromic material that may be included in an electrochromic device to configure the electrochromic device to achieve more effective color display control. It will be understood that the “visible light region” refers to the entire wavelength spectrum of visible light, also referred to as the visible spectrum (e.g., about 400 nm to about 700 nm).

For example, in the compound represented by Chemical Formula 1, the following electrochemical reaction may reversibly occur depending on the applied voltage.

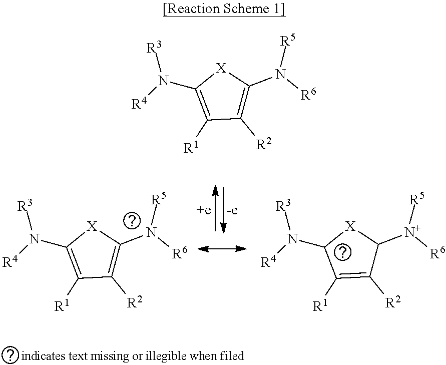

For example, X may be S, Se, or Te.

For example, R¹ and R² may each independently be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof. R¹ and R² may each independently be present or linked to each other to form a ring.

For example, R¹ and R² may each independently be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, or any combination thereof. R¹ and R² may each independently be present or linked to each other to form a ring.

For example, one of R¹ or R² may be hydrogen and the other of R¹ or R² may be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof.

For example, one of R¹ or R² may be hydrogen and the other of R¹ or R² may be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, or any combination thereof.

For example, at least one of R³ to R⁶ may include a reaction site for the electro-polymerization reaction described below.

For example, at least one of R³ or R⁴ may be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group.

For example, R³ and R⁴ may each independently be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group.

For example, at least one of R⁵ or R⁶ may be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group.

For example, R⁵ and R⁶ may each independently be a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group.

For example, the compound may be represented by Chemical Formula 1A, depending on the substituent.

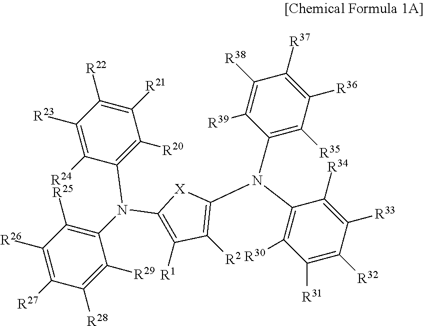

In Chemical Formula 1A,
X, R¹, and R² are the same as described above, such that X may be O, S, Se, or Te; and R¹ and R² may each independently be hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof; at least one of R¹ or R² may be a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof; and R¹ and R² may each independently be present or may be linked to each other to form a ring,
R²⁰ to R³⁹ may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, a hydroxyl group, a halogen, or any combination thereof, and
R²⁰ to R³⁹ may each independently be present or two adjacent ones of R²⁰ to R³⁹ may be linked to each other to form a ring.

For example, R²⁰ to R³⁹ may each independently be hydrogen or a substituted or unsubstituted C1 to C30 alkyl group.

For example, in Chemical Formula 1 and/or Chemical Formula 1A, R¹ and R² may be linked to each other to form a ring, wherein the ring may be, for example, a substituted or unsubstituted C6 to C30 aromatic ring, a substituted or unsubstituted C3 to C30 aliphatic ring, a substituted or unsubstituted C3 to C30 heterocycle, or any combination thereof.

For example, in Chemical Formula 1A, two adjacent ones of R²⁰ to R³⁹ may be linked to each other to form a ring, wherein the ring may be, for example, a substituted or unsubstituted C6 to C30 aromatic ring, a substituted or unsubstituted C3 to C30 aliphatic ring, a substituted or unsubstituted C3 to C30 heterocycle, or any combination thereof.

For example, the compound may be represented by Chemical Formula 1B-1 or Chemical Formula 1B-2, depending on the core structure.

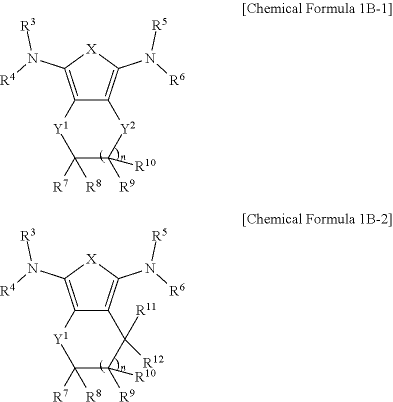

In Chemical Formula 1B-1 and Chemical Formula 1B-2,
X, and R³ to R⁶ may be the same as described above, such that X may be O, S, Se, or Te; R³ to R⁶ may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof; at least one of R³ or R⁴ may be a substituted or unsubstituted C6 to C30 aryl group; and at least one of R⁵ or R⁶ may be a substituted or unsubstituted C6 to C30 aryl group,
Y¹ and Y² may each independently be O, S, Se, or Te,
R⁷ to R¹² may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
R³ to R¹² may each independently be hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
R⁷ to R¹² may each independently be present or two adjacent ones of R⁷ to R¹² may be linked to each other to form a ring, and
n may be an integer of 0 to 3.

For example, Y¹ and Y² may be the same as or different from X and for example, X may be S, Se, or Te and Y¹ and Y² may each be O.

For example, R⁷ to R¹² may each independently be hydrogen or a substituted or unsubstituted C1 to C30 alkyl group.

For example, n may be 1, 2, or 3.

As an example, the compound may be represented by Chemical Formula 1C-1 or Chemical Formula 1C-2.

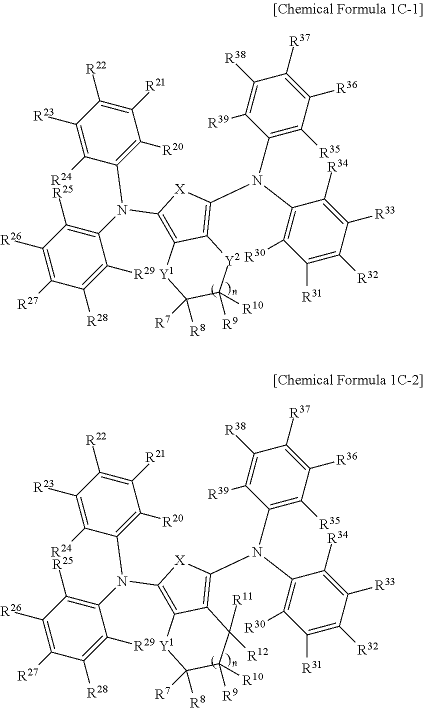

In Chemical Formula 1C-1 and Chemical Formula 1C-2, X, Y¹, Y², R⁷ to R¹², R²⁰ to R³⁹, and n may be the same as described above. For example, X, Y¹, and Y² may each independently be O, S, Se, or Te; R⁷ to R¹² and R²⁰ to R³⁹ are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof; R⁷ to R¹² may each independently be present or two adjacent ones of R⁷ to R¹² may be linked to each other to form a ring; R²⁰ to R³⁹ may each independently be present or two adjacent ones of R²⁰ to R³⁹ may be linked to each other to form a ring; and n may be an integer of 0 to 3.

The compound may be, for example one of the compounds listed in Group 1, but is not limited thereto.

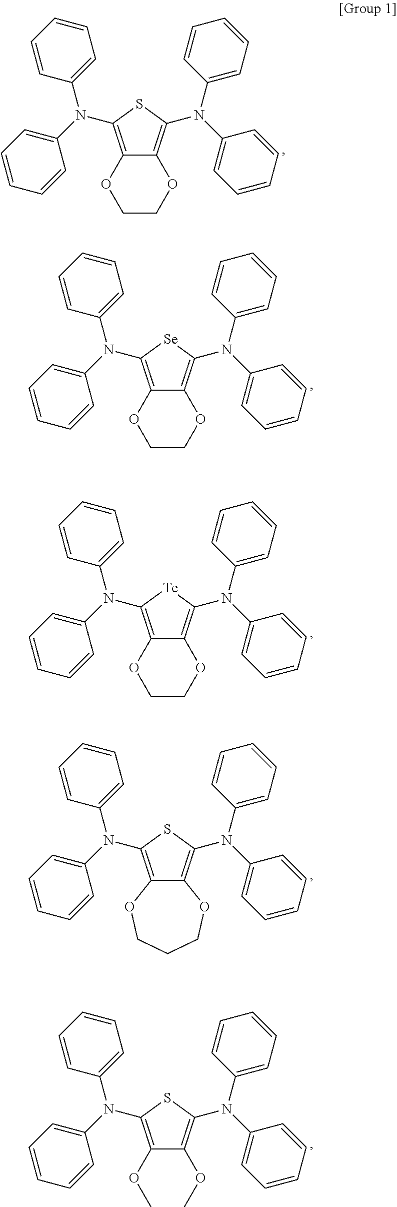

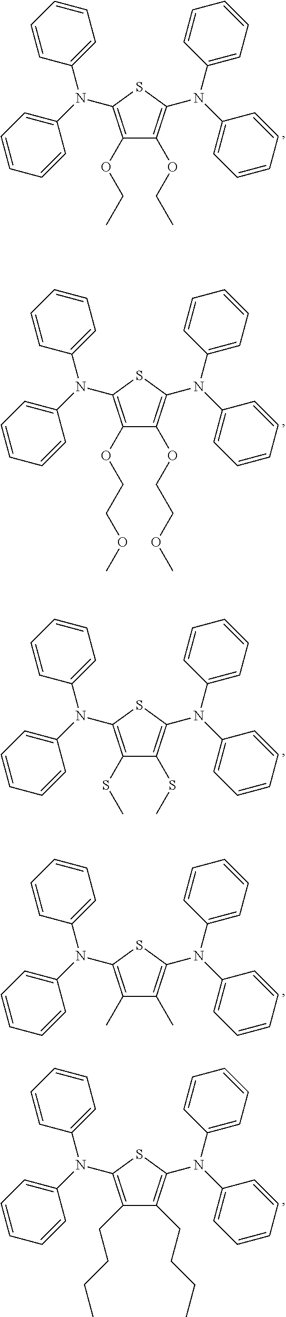

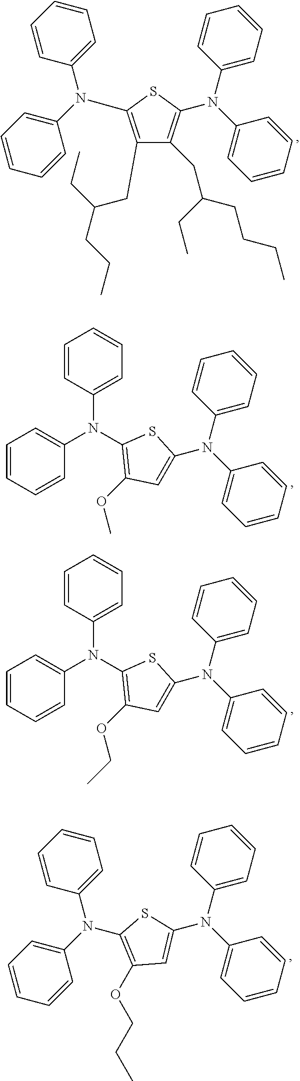

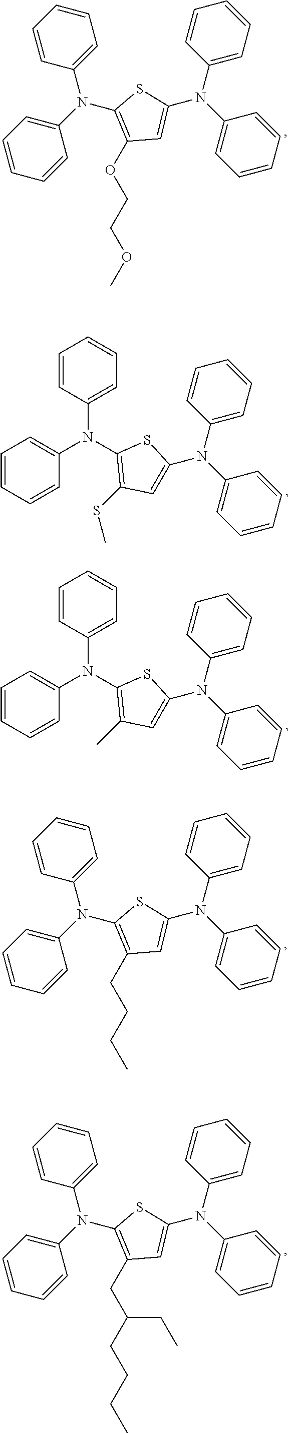

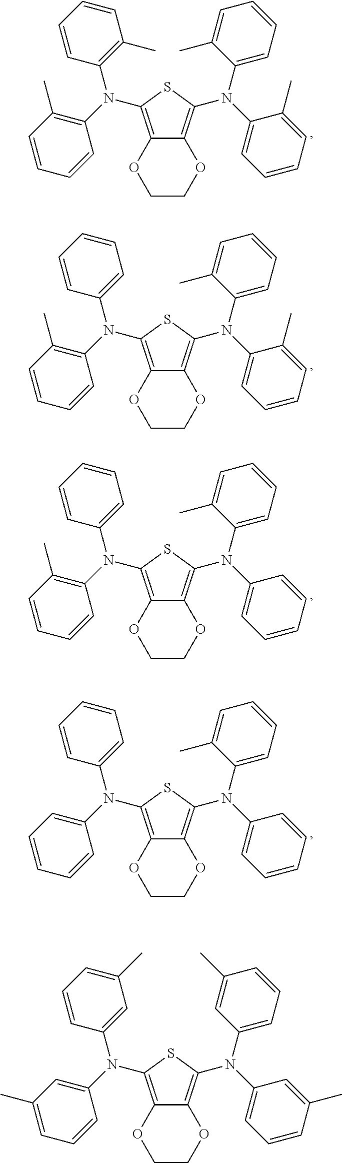

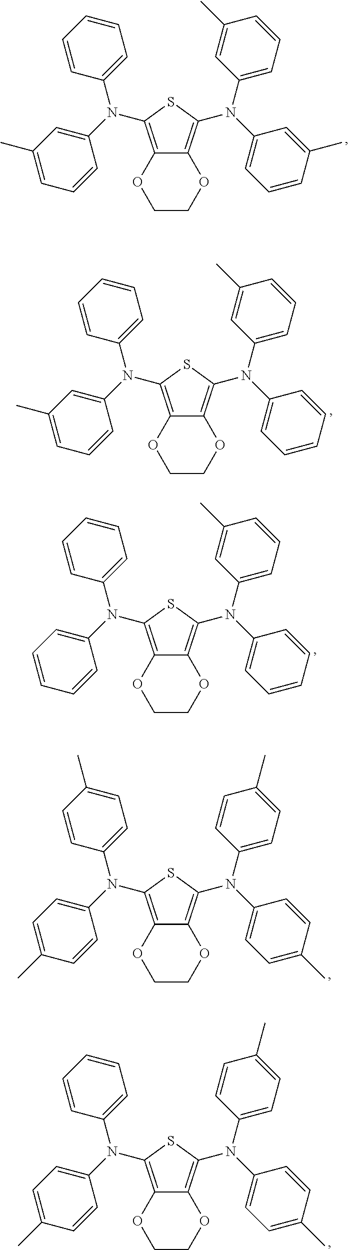

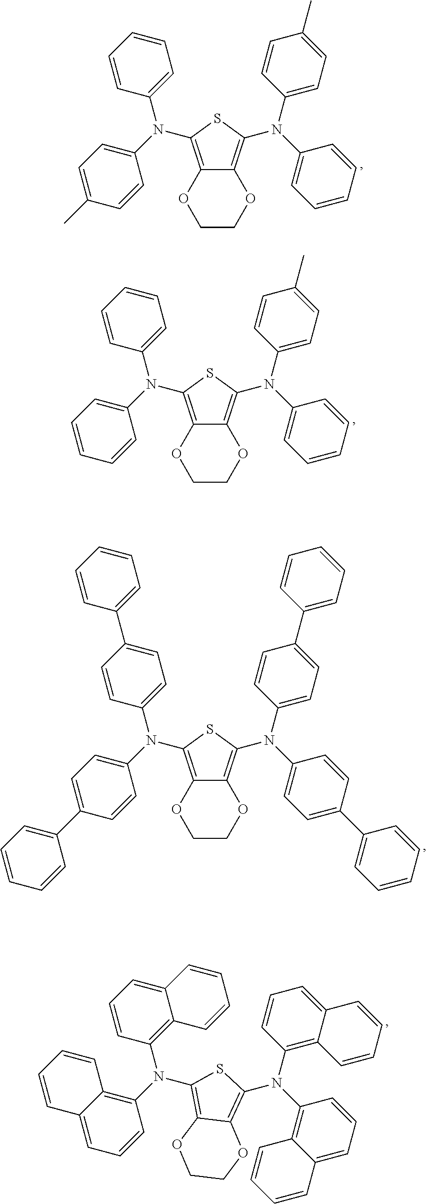

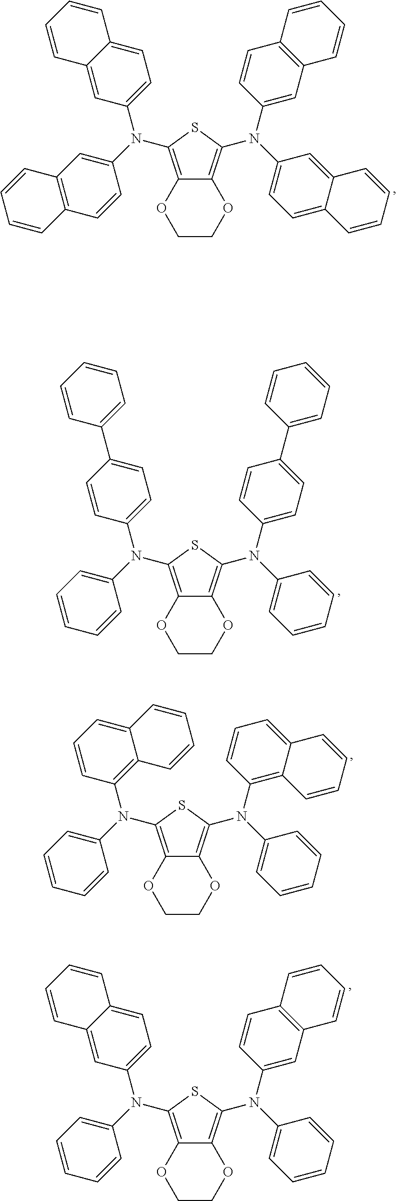

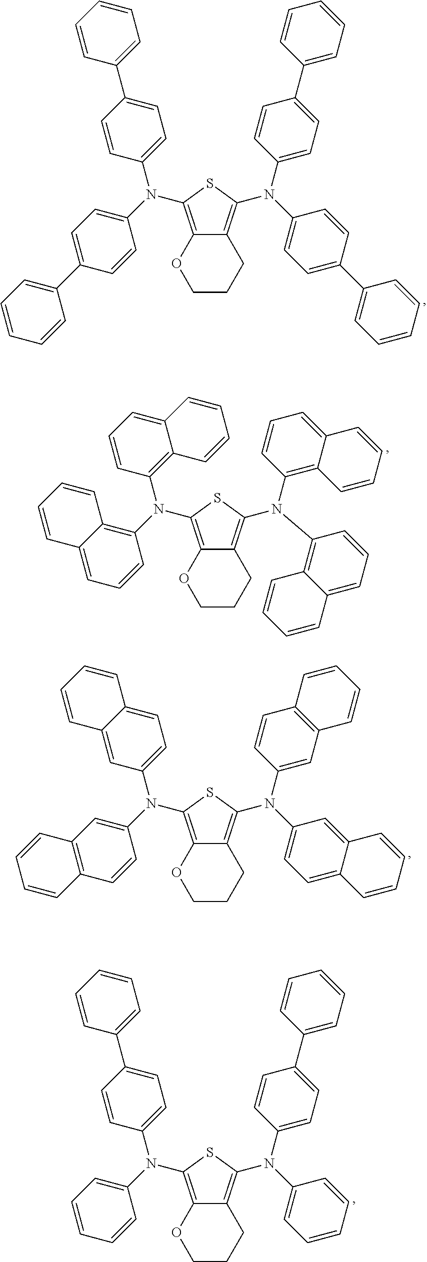

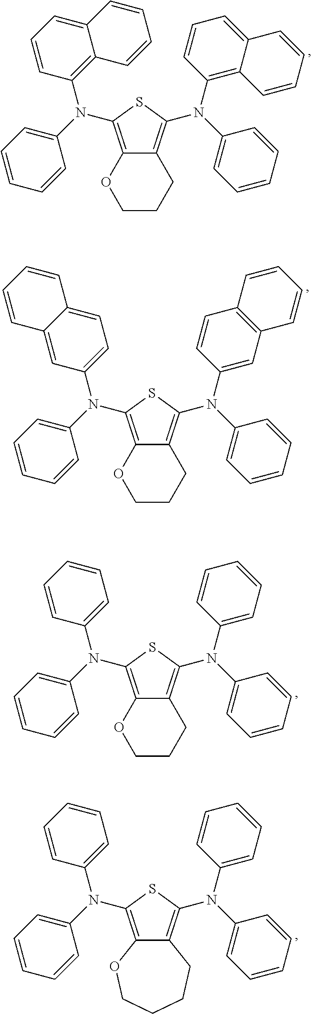

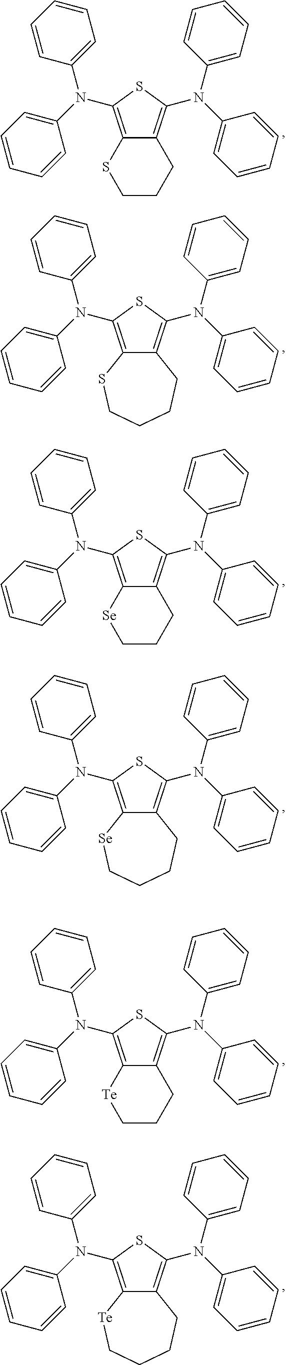

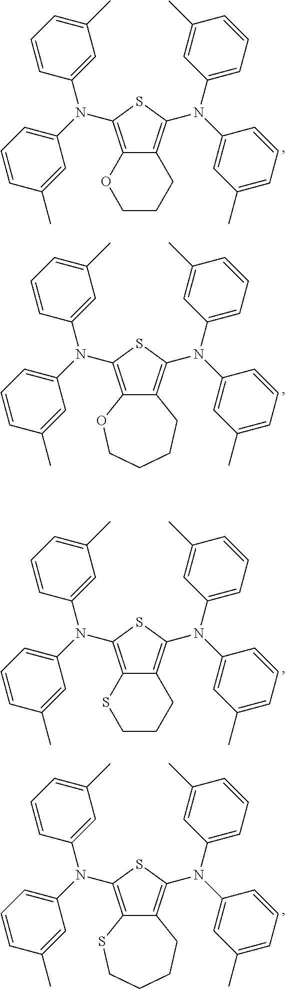

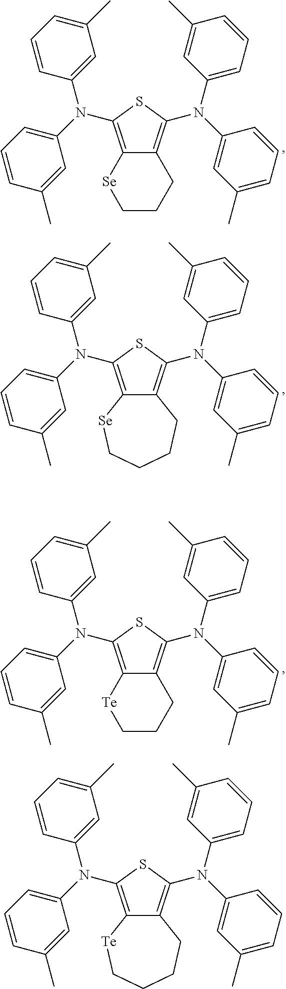

The above-described compound may be polymerized to form a polymer, and the polymer may include a structural unit derived from the compound.

The polymer may be a resulting material obtained by electro-polymerization using a substituent of the conjugated structure of the above-described compound as a reaction site with a substituent of the conjugated structure of an adjacent compound. For example, a substituted or unsubstituted aryl group, which is at least one of R³ or R⁴ in Chemical Formula 1, 1B-1, or 1B-2, and a substituted or unsubstituted aryl group, which is at least one of R⁵ or R⁶ in Chemical Formula 1, 1B-1, or 1B-2, may be polymerization reaction sites. For example, at least some of the substituted or unsubstituted phenyl groups of the compound represented by Chemical Formula 1A, 1C-1, or 1C-2 may be polymerization reaction sites.

For example, in a compound of Chemical Formula 1 in which R³, R⁴, R⁵, and R⁶ are each a phenyl group, as shown in Reaction Scheme 2, a radical cation is generated in the compound by an oxidation reaction, and as shown in Reaction Scheme 3, a coupling occurs between radical cations of adjacent compounds, and a reaction may occur toward forming a longer conjugated structure, as the proton at the position where the coupling occurs is removed. By these successive reactions, compounds may be formed into multimers such as dimers, which may then be further expanded to form polymers.

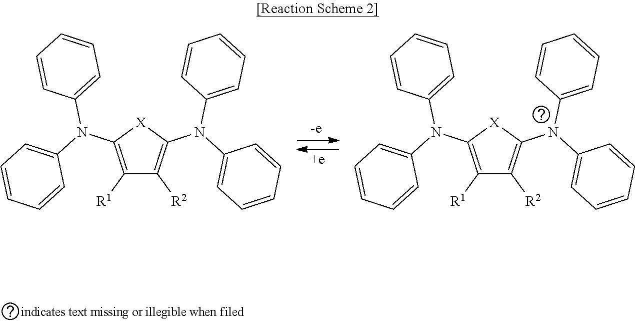

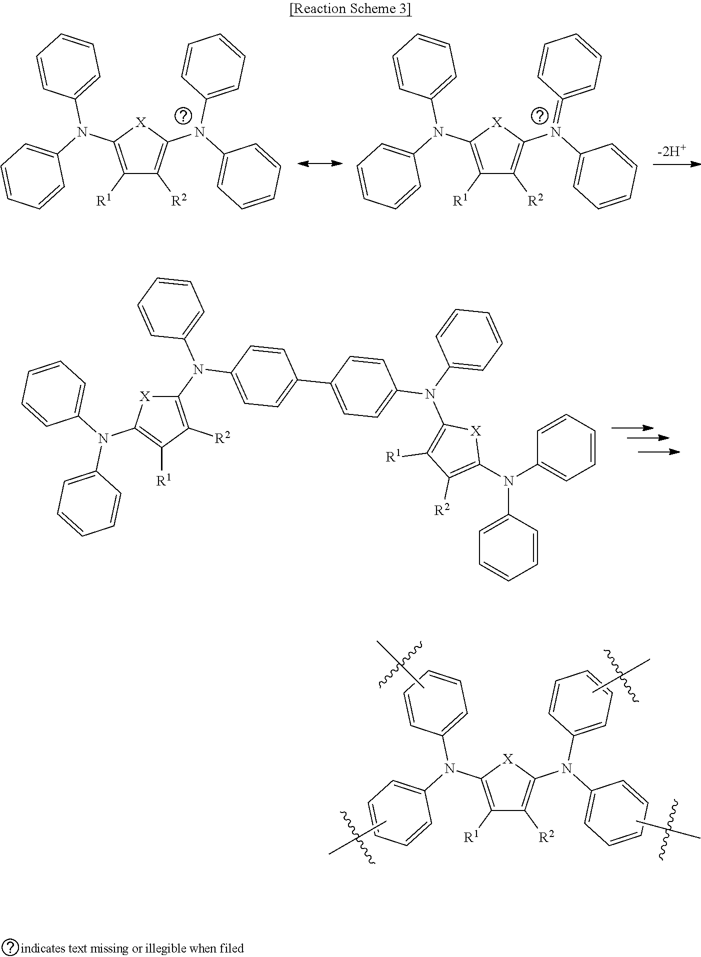

The polymer obtained by electro-polymerization in this way may exhibit improved electrochromic properties because their optical properties may be reversibly changed by rapid electrochemical oxidation and reduction reactions, thereby enabling the polymer to be used to form an electrochromic device configured to reversibly convert to a colored state to be configured to absorb at least a portion of light in the visible light region or a bleached state to be configured to not absorb light in the visible light region, including transmitting the portion of light that is absorbed in the colored state (e.g., to transmit light in the entirety or substantially the entirety of the visible light region), for example to reversibly convert between the colored state and the bleached state, at an improved speed. As a result, an electrochromic device may be configured to exhibit improved color-changing display performance, including more effective color display control, based on including the polymer.

For example, the polymer may be obtained through oxidative polymerization by coating a solution containing the above-described compound and adding an oxidizing agent. Thin films (polymer thin films) including such a polymer may exhibit high thin film stability and uniformity. The polymer thin film may include a polymer, a non-polymerized compound, or any combination thereof.

For example, the above-described compound and/or polymer may be configured to be reversibly converted into and/or between a colored state in which the above-described compound and/or polymer absorbs or is configured to absorb at least a portion of light in the visible light region (e.g., one or more wavelength bands within the visible light region) to display a color (e.g., display black) based on a first voltage being applied to the above-described compound and/or polymer, and a bleached state in which the above-described compound and/or polymer transmits or is configured to transmit light in the visible light region, including transmitting the portion of light absorbed in the colored state, to display no color (e.g., display transparency) based on a second voltage being applied to the above-described compound and/or polymer (that is different from the first voltage). For example, the above-described compound and/or polymer may be configured to be reversibly converted, based on an applied voltage, into the colored state or the bleached state (e.g., configured to be reversibly converted, based on an applied voltage, between the colored state and the bleached state). The first and second voltages may be different voltages, including where one of the first and second voltages may be a zero voltage (e.g., 0 V) although example embodiments are not limited thereto, such that the first and second voltages may be different ones of 0 V, 1 V, 1.5 V, 2 V, 2.5 V, or 3 V.

For example, the above-described compound and/or polymer may be configured to be reversibly converted into a colored state in which the above-described compound and/or polymer absorbs or is configured to absorb at least a portion of light in the visible light region (e.g., to absorb light in one or more limited wavelength regions in the visible light region) to display a color based on an application of a first voltage (e.g., a nonzero voltage, for example one of 1 V, 1.5 V, 2 V, 2.5 V, or 3 V) to the above-described compound and/or polymer, and the above-described compound and/or polymer may be configured to be reversibly converted into a bleached state in which the above-described compound and/or polymer transmits or is configured to transmit light in the visible light region (e.g., to transmit the portion of light absorbed in the colored state, to transmit light in all or substantially all of the visible light region, or any combination thereof) to display no color based on no voltage (e.g., a second voltage that is zero voltage, or 0 V) being applied to the above-described compound and/or polymer.

For example, the above-described compound and/or polymer may be configured to be reversibly converted into a colored state in which the above-described compound and/or polymer absorbs or is configured to absorb at least a portion of light in the visible light region to display a color based on no voltage (e.g., a first voltage that is zero voltage, or 0 V) being applied to the above-described compound and/or polymer, and the above-described compound and/or polymer may be configured to be reversibly converted into a bleached state in which the above-described compound and/or polymer transmits or is configured to transmit light in the visible light region (e.g., to transmit the portion of light absorbed in the colored state, to transmit light in all or substantially all of the visible light region, or any combination thereof) to display no color based on application of a second voltage that is different from the first voltage (e.g., a nonzero voltage, e.g., one of 1 V, 1.5 V, 2 V, 2.5 V, or 3 V) to the above-described compound and/or polymer.

The above-described compound and/or polymer may include one or more electrochromic materials that are configured to reversibly display opaque (e.g., display black) or transparent (e.g., display transparency) according to an applied voltage. For example, the above-described compound and/or polymer may include one or more anodic coloration materials that are configured to display a color (including black) in an oxidized state and become transparent in a reduced state. For example, the above-described compound and/or polymer may include one or more cathodic coloration materials that are configured to display a color (including black) in a reduced state and become transparent in an oxidized state.

For example, the above-described compound and/or polymer may be configured to exhibit a difference in light transmittance of greater than or equal to about 30% at a particular (or, alternatively, predetermined) wavelength within the visible light wavelength region, according to the applied voltage. For example, the above-described compound and/or polymer may be configured to exhibit a difference in light transmittance at a particular wavelength (e.g., a wavelength of 470 nm) based on a difference in an applied voltage that is applied to the electrochromic device, the difference in light transmittance about 30% to about 100%. For example, where the above-described compound and/or polymer (or an electrochromic material and/or device including same) is described to exhibit a difference in light transmittance according to applied voltage, the above-described compound and/or polymer (or an electrochromic material and/or device including same) may be configured to exhibit a first transmittance at the particular wavelength based on a first voltage being applied thereto and to exhibit a second transmittance at the particular wavelength based on a second voltage being applied thereto, where the second voltage is different from the first voltage, the difference between the first and second transmittances is the aforementioned difference in light transmittance at the particular wavelength and may be greater than or equal to about 30% (e.g., about 30% to about 100%), where the first and second voltages may be different voltages, including where one of the first and second voltages may be a zero voltage (e.g., 0 V) although example embodiments are not limited thereto, such that the first and second voltages may be different ones of 0 V, 1 V, 1.5 V, 2 V, 2.5 V, or 3 V. Accordingly, the above-described compound and/or polymer may be configured to exhibit a difference in light transmittance at a wavelength of 470 nm based on a difference in an applied voltage that is applied to the electrochromic device, where the difference in light transmittance may be about 30% to about 100%. For example, the difference in light transmittance at a wavelength of about 470 nm according to the applied voltage of the above-described compound and/or polymer (e.g., the difference between a first transmittance based on application of a first voltage and a second transmittance exhibited by the above-described compound and/or polymer based on application of a second, different voltage thereto) may be greater than or equal to about 30%, and within the range of about 30% to about 100%, about 35% to about 100%, about 40% to about 100%, about 45% to about 100%, about 50% to about 100%, about 55% to about 100%, or about 60% to about 100%.

The above-described compound and/or polymer may be applied to (e.g., included in) various devices and electronic devices requiring and/or having electrochemical properties.

As an example, the compound and/or polymer may be applied as one or more electrochromic materials in electrochromic devices that require and/or have electrochemical and optical properties.

An electrochromic device according to some example embodiments is described below.

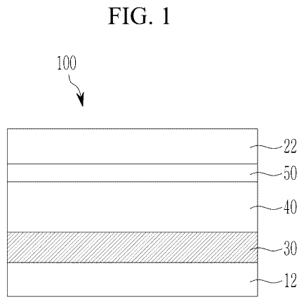

FIG. 1 is a cross-sectional view illustrating an electrochromic device according to some example embodiments.

Referring to FIG. 1, an electrochromic device 100 according to some example embodiments includes a first electrode 12 and a second electrode 22 facing each other, and a first electrochromic layer 30 between the first electrode 12 and the second electrode 22, and a first electrolyte 40 in contact with the first electrochromic layer 30 between the first electrode 12 and the second electrode 22. The electrochromic device 100 is shown to further include a first ion storage layer 50, but example embodiments are not limited thereto and in some example embodiments the first ion storage layer 50 may be omitted from the electrochromic device 100.

A substrate (not shown) may be disposed on one surface of the first electrode 12 and/or the second electrode 22. The substrate may be made of transparent glass or a polymer, and the polymer may include one or more selected from, for example, polyacrylate, polyethylene phthalate, polyethylene naphthalate, polycarbonate, polyarylate, polyether imide, polyether sulfone, and/or polyimide.

One of the first electrode 12 or the second electrode 22 may be a working electrode and the other may be a counter electrode that is configured to maintain the flow of electrons. At least one of the first electrode 12 or the second electrode 22 may be made of a transparent conductor, and each of the first electrode 12 and the second electrode 22 may include an inorganic conductive material such as indium tin oxide (ITO), fluorine doped tin oxide (FTO), antimony doped tin oxide (ATO), nickel oxide (NiO), titanium oxide (TiO₂), or any combination thereof, or an organic conductive material such as polyacetylene, polythiophene, or a derivative thereof.

The first electrochromic layer 30 includes an electrochromic material (also referred to herein interchangeably as a first electrochromic material). The electrochromic material may be a compound that may be configured to reversibly display color or change color by electrochemical change depending on the direction of an electric field when voltage is applied to the electrochromic material (e.g., based on application of voltage to the first electrochromic layer 30), and may include the above-described compound (e.g., the compound represented by Chemical Formula 1), polymer (e.g., polymer including a structural unit derived from the compound), or any combination thereof. In example embodiments where the electrochromic material includes the above-described polymer including a structural unit derived from the above-described compound, the polymer may be a reaction product of electro-polymerization using at least one of R³ to R⁶ of Chemical Formula 1 as a reaction site.

The electrochromic material may include one or more cathodic coloration materials or anodic coloration materials. The cathodic coloration materials are materials that are configured to display a color (including black) in a reduced state and become transparent in an oxidized state, and anodic coloration materials are materials that are configured to display a color (including black) in an oxidized state and become transparent in a reduced state.

For example, the electrochromic materials may be adsorbed onto semiconductor nanoparticles. The semiconductor nanoparticles may include titanium oxide (TiO₂), zinc oxide (ZnO₂), tungsten oxide (WO₃), or any combination thereof, but example embodiments are not limited thereto. The shape of the semiconductor nanoparticles may be various, such as sphere, tetrahedron, cylinder, triangle, disk, tripod, tetrapod, cube, box, star, and tube, and the size (average particle diameter or major diameter) of the semiconductor nanoparticles may be about 1 nm to about 100 nm.

An adhesive auxiliary layer (not shown) may be further included between the first electrode 12 and the first electrochromic layer 30 to improve the adhesion between the first electrode 12 and the first electrochromic layer 30. However, the adhesive auxiliary layer may be omitted.

The first electrolyte 40 may be between (e.g., may be filled between) the first electrode 12 and the second electrode 22 and in contact with the first electrochromic layer 30. The first electrolyte 40 may supply substances that promote an oxidation/reduction reaction of the electrochromic material and may include a liquid electrolyte or a solid polymer electrolyte. The liquid electrolyte or solid polymer electrolyte may be an ionic substance, and as the ionic liquid electrolyte, a solution in which lithium salts such as LiOH or LiClO₄, potassium salts such as KOH, and sodium salts such as NaOH or NaClO₄ are dissolved in a solvent may be used, but is not limited thereto. Examples of the ionic solid electrolyte that may be used may include poly(2-acrylamino-2-methylpropane sulfonic acid), poly(ethylene oxide), 1-butyl-3-methylimidazolium bis(trifluoromethylsulfonyl)imide (BMIMTFSI), and/or 1-butyl-3-methylimidazolium tetrafluoroborate (BMIMMBF₄), but are not limited thereto.

Although the first electrolyte 40 is shown in FIG. 1 as a layer independent of the first electrochromic layer 30, it will be understood that this is merely a schematic view, and the present disclosure is not limited thereto. In the present disclosure, an expression “a first electrolyte 40 in contact with the first electrochromic layer 30” may include the following cases:

In some cases, the first electrochromic layer 30 is a solid, the first electrolyte 40 is a solid, and a surface of the first electrolyte 40 contacts with a surface of the first electrochromic layer 30.

In some cases, the first electrochromic layer 30 is a solid, the first electrolyte 40 is a liquid, and the first electrolyte 40 contacts with a surface of the first electrochromic layer 30.

In some cases, the first electrolyte 40 is mixed in the first electrochromic layer 30.

In a case where the first electrolyte 40 is mixed in the first electrochromic layer 30, the first electrolyte 40 may be in contact with and (uniformly) mixed with the above-described electrochromic material (the first electrochromic material). In addition, the first electrolyte 40 may be in a liquid or solid form, such as in a liquid form.

In a case where the first electrolyte 40 is mixed in the first electrochromic layer 30, the electrochromic device 100 may also be described as including a first electrode 12 and a second electrode 22 facing each other, and a first electrochromic layer 30 between the first electrode 12 and the second electrode 22, wherein the first electrochromic layer 30 comprises the above-described electrochromic material (the first electrochromic material) and the first electrolyte 40. In the first electrochromic layer 30, the first electrolyte 40 may be in contact with and (uniformly) mixed with the above-described electrochromic material (the first electrochromic material). The first electrochromic layer 30 may be a liquid layer or a solid layer, such as a liquid layer. The electrochromic device 100 may further comprise a first ion storage layer 50. The first ion storage layer 50 may be between first electrochromic layer 30 and the second electrode 22.

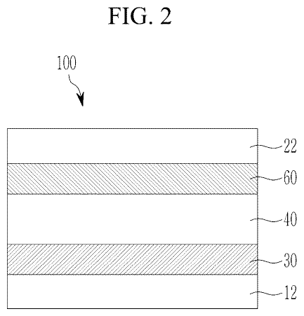

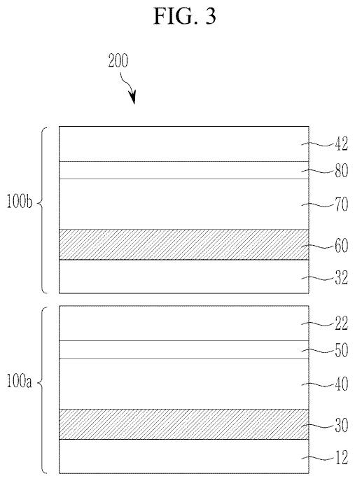

The above description with respect to “a first electrolyte 40 in contact with the first electrochromic layer 30” may also be applicable to FIGS. 2 and 3 to be described later.

The first ion storage layer 50 may perform electrochemical oxidation and/or reduction reactions to maintain charge balance through the first electrolyte 40. The first ion storage layer 50 may include, for example, a metal oxide, and may include an oxide of titanium (Ti), vanadium (V), niobium (Nb), tantalum (Ta), molybdenum (Mo), tungsten (W), cobalt (Co), rhodium (Rh), iridium (Ir), nickel (Ni), chromium (Cr), manganese (Mn), iron (Fe), aluminum (Al), titanium (Ti), or any combination thereof, but is not limited thereto.

An electrochromic device 100 including the above-described compound and/or polymer as an electrochromic material may be configured to reversibly display opaque (colored state, for example display black) or transparent (bleached state) (e.g., reversibly convert between displaying opaque and displaying transparent) depending on an applied voltage (e.g., an applied voltage that is applied to the electrochromic material of the electrochromic layer 30). For example, the electrochromic device 100 may be configured to reversibly display opaque based on a first voltage being applied to the electrochromic material via the first and second electrodes 12 and 22 and to reversibly display transparent based on a second, different voltage being applied to the electrochromic material via the first and second electrodes 12 and 22. The first and second voltages may be different voltages, including where one of the first and second voltages may be a zero voltage (e.g., 0 V) although example embodiments are not limited thereto, such that the first and second voltages may be different ones of 0 V, 1 V, 1.5 V, 2 V, 2.5 V, or 3 V. For example, the electrochromic device 100 may be configured to exhibit a difference in light transmittance at a particular wavelength in the visible light region (e.g., a wavelength greater than or equal to about 400 nm and less than or equal to about 700 nm, for example a wavelength of about 470 nm) depending on a difference in an applied voltage of the electrochromic device 100 (e.g., a difference in an applied voltage that is applied to the electrochromic device 100, for example applied to the electrochromic material of the electrochromic layer 30 via the first and second electrodes 12 and 22). For example, the electrochromic device 100 may be configured to exhibit a first transmittance at the particular wavelength in the visible light region based on a first voltage being applied to the electrochromic device 100 (e.g., applied to the electrochromic layer 30 via the first and second electrodes 12 and 22) and to exhibit a second transmittance at the particular wavelength in the visible light region based on a second voltage being applied to the electrochromic device 100 (e.g., applied to the electrochromic layer 30 via the first and second electrodes 12 and 22), where the second voltage is different from the first voltage, and a difference between the first and second transmittances is the aforementioned difference in light transmittance exhibited by the electrochromic layer 30 (and thus the electrochromic device 100) at the particular wavelength in the visible light region and may be greater than or equal to about 30% (e.g., about 30% to about 100%). The first and second voltages may be different voltages, including where one of the first and second voltages may be a zero voltage (e.g., 0 V) although example embodiments are not limited thereto, such that the first and second voltages may be different ones of 0 V, 1 V, 1.5 V, 2 V, 2.5 V, or 3 V. The difference in light transmittance exhibited by the electrochromic layer 30 (and thus the electrochromic device 100) at the particular wavelength in the visible light region may be greater than or equal to about 30%, and within the above range may be about 30% to about 100%, about 35% to about 100%, about 40% to about 100%, about 45% to about 100%, about 50% to about 100%, about 55% to about 100%, or about 60% to about 100%. Accordingly, the electrochromic device 100 may be configured to exhibit a difference in light transmittance at a wavelength of 470 nm based on a difference in an applied voltage that is applied to the electrochromic device 100, where the difference in light transmittance may be about 30% to about 100%.

An electrochromic device according to some example embodiments is described below.

FIG. 2 is a cross-sectional view illustrating an electrochromic device according to some example embodiments.

Referring to FIG. 2, an electrochromic device 100 according to some example embodiments includes, similar to the above-described example embodiments shown in FIG. 1, a first electrode 12, a second electrode 22, a first electrochromic layer 30, and a first electrolyte 40.

However, unlike some example embodiments including the example embodiments shown in FIG. 1, the electrochromic device 100 according to the some example embodiments, including the example embodiments shown in FIG. 2, further includes a second electrochromic layer 60, in addition to the first electrochromic layer 30, between the first electrode 12 and the second electrode 22. That is, the first electrochromic layer 30 may be close to the first electrode 12 between the first electrode 12 and the second electrode 22, and the second electrochromic layer 60 may be close to the second electrode 22 between the first electrode 12 and the second electrode 22, for example such that the first electrochromic layer 30 is between the second electrochromic layer 60 and the first electrode 12, and the second electrochromic layer 60 is between the first electrochromic layer 30 and the second electrode 22. The first electrochromic layer 30 and the second electrochromic layer 60 may each be in contact with the first electrolyte 40.

The first electrochromic layer 30 is as described above with reference to the example embodiments shown in FIG. 1 and may include the above-described compound, polymer, or any combination thereof (hereinafter referred to as “first electrochromic material”).

The second electrochromic layer 60 may replace the above-described first ion storage layer 50 or may be included in the first ion storage layer 50. The second electrochromic layer 60 may include a second electrochromic material that is different from the first electrochromic material. The second electrochromic material may include an inorganic material, an organic material, and/or an inorganic-organic material, and may include a reduction chromophore material or an oxidation chromophore material different from the first electrochromic material.

The second electrochromic material, like the first electrochromic material, may be configured to be reversibly converted into a colored state (opaque) wherein the second electrochromic material absorbs and/or is configured to absorb at least a portion of light in the visible light region to display a color, or a bleached state (transparent) wherein the second electrochromic material transmits and/or is configured to transmit light in the visible light region (e.g., configured to be reversibly converted between the color stated and the bleached state), depending on an applied voltage.

For example, the second electrochromic material may have complementary chromophore properties to the first electrochromic material. For example, the light absorption state (opaque, colored state) of the first electrochromic material by the reduction reaction and the light absorption state (opaque, colored state) of the second electrochromic material by the oxidation reaction occur simultaneously or substantially simultaneously, so that the electrochromic device 100 may be configured to display opaque (colored state). For example, the light transmitting state (transparent, bleached state) of the first electrochromic material by the oxidation reaction and the light transmitting state (transparent, bleached state) of the second electrochromic material by the reduction reaction are simultaneously or simultaneously achieved, so that the electrochromic device 100 may be configured to display transparent (bleached state).

The second electrochromic material may be, for example, a metal oxide, such as LiNiO₂, IrO₂, NiO, V₂O₅, LixCoO₂, Rh₂O₃, CrO₃, WO₃, MoO₃, Nb₂O₅, Ta₂O₅, TiO₂, or any combination thereof.

The second electrochromic material may be, for example, an organic material and may be a material represented by Chemical Formula A.

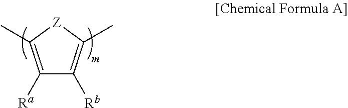

In Chemical Formula A,
Z may be O, S, Se, or Te,
R^{a} and R^{b} may each independently be hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
R^{a} and R^{b} may each independently be present or linked to each other to form a ring, and
m is the number of repeating units and may be, for example, 2 to 100.

The second electrochromic material may have different optical properties than the first electrochromic material.

For example, when the second electrochromic material is in a colored state (opaque), the absorption characteristics of the second electrochromic material may be different from the absorption characteristics of the first electrochromic material, and for example, a maximum absorption wavelength (λₘₐₓ₂) in the visible light absorption spectrum of the second electrochromic material may be different from a maximum absorption wavelength (λₘₐₓ₁) in the visible light absorption spectrum of the first electrochromic material. For example, the first electrochromic material may be configured to exhibit a first maximum absorption wavelength (λₘₐₓ₁) in the visible light region based on being reversibly converted to a respective colored state of the first electrochromic material to be configured to absorb at least a portion of light in a visible light region, the second electrochromic material may be configured to exhibit a second maximum absorption wavelength (λₘₐₓ₂) in the visible light region based on being reversibly converted to a respective colored state of the second electrochromic material to be configured to absorb at least a portion of light in a visible light region, and the second maximum absorption wavelength of the second electrochromic material may be different from the first maximum absorption wavelength of the first electrochromic material. For example, the maximum absorption wavelength (λₘₐₓ₁) in the visible light absorption spectrum of the first electrochromic material may be between about 400 nm and 550 nm, and the maximum absorption wavelength (λₘₐₓ₂) in the visible light absorption spectrum of the second electrochromic material may be greater than or equal to about 500 nm and less than about 750 nm.

The light absorption characteristics in a colored state of the electrochromic device 100 including the first electrochromic layer 30 and the second electrochromic layer 60 may be the same as a combination of visible light absorption spectra of the first and second electrochromic materials, for example, the electrochromic device 100 may exhibit light absorption characteristics over a wavelength region of about 400 nm to about 650 nm, for example, display black (e.g., display the color black).

The electrochromic device 100 including the first electrochromic layer 30 and the second electrochromic layer 60 may be configured to reversibly display black (e.g., absorb all or substantially all visible light) or transparency (e.g., transmit all or substantially all visible light) according to (e.g., based on) the applied voltage. For example, the electrochromic device 100 including the first electrochromic layer 30 and the second electrochromic layer 60 may be configured to exhibit a difference in light transmittance at a particular wavelength in the visible light region (e.g., a wavelength greater than or equal to about 400 nm and less than or equal to about 700 nm, for example a wavelength of about 470 nm) depending on a difference in an applied voltage of the electrochromic device 100 (e.g., a difference in an applied voltage that is applied to the electrochromic device 100, for example applied to the electrochromic material of the electrochromic layer 30 via the first and second electrodes 12 and 22). For example, the electrochromic device 100 including the first electrochromic layer 30 and the second electrochromic layer 60 may be configured to exhibit a first transmittance at a particular wavelength in the visible light region (e.g., a wavelength greater than or equal to about 400 nm and less than or equal to about 700 nm, for example a wavelength of about 470 nm) based on a first voltage being applied thereto (e.g., applied to both the first and second electrochromic layers 30 and 60 via first and second electrodes 12 and 22) and to exhibit a second transmittance at the particular wavelength in the visible light region based on a second voltage being applied thereto (e.g., applied to both the first and second electrochromic layers 30 and 60 via first and second electrodes 12 and 22), where the second voltage is different from the first voltage, and a difference between the first and second transmittances is a difference in light transmittance that the electrochromic device 100 (including the first electrochromic layer 30 and the second electrochromic layer 60) is configured to exhibit at the particular wavelength in the visible light region and may be greater than or equal to about 30% (e.g., about 30% to about 100%). The first and second voltages may be different voltages, including where one of the first and second voltages may be a zero voltage (e.g., 0 V) although example embodiments are not limited thereto, such that the first and second voltages may be different ones of 0 V, 1 V, 1.5 V, 2 V, 2.5 V, or 3 V. Such a difference in light transmittance that the electrochromic device 100 (including the first electrochromic layer 30 and the second electrochromic layer 60) is configured to exhibit at the particular wavelength in the visible light region may be greater than or equal to about 30% in the visible light region (e.g., greater than or equal to about 400 nm and less than or equal to about 700 nm) according to the applied voltage and within the above range, about 30% to about 100%, about 35% to about 100%, about 40% to about 100%, about 45% to about 100%, about 50% to about 100%, about 55% to about 100%, or about 60% to about 100%. Accordingly, the electrochromic device 100 (including the first electrochromic layer 30 and the second electrochromic layer 60) may be configured to exhibit a difference in light transmittance at a wavelength of 470 nm based on a difference in an applied voltage that is applied to the electrochromic device 100, where the difference in light transmittance may be about 30% to about 100%.

Hereinafter, the electrochromic device according to some example embodiments is illustrated below.

FIG. 3 is a cross-sectional view illustrating an electrochromic device according to some example embodiments.

FIG. 3 provides a stacked electrochromic device 200 in which a plurality of electrochromic devices 100a and 100b are stacked. The stacked electrochromic device 200 includes a first electrochromic device 100a and a second electrochromic device 100b stacked on the first electrochromic device 100a, where the first electrochromic device 100a may include (or may be) the electrochromic device 100 as described above with reference to FIGS. 1 and 2.

The first electrochromic device 100a includes, as described above, the first electrode 12 and the second electrode 22 facing each other, a first electrochromic layer 30 between the first electrode 12 and the second electrode 22, a first electrolyte 40 between the first electrode 12 and the second electrode 22 and contacting to the first electrochromic layer 30, and a first ion storage layer 50. The first electrochromic layer 30 includes the above-described first electrochromic material, which may include one or more compounds, polymers, or any combination thereof.

The second electrochromic device 100b includes a third electrode 32 and a fourth electrode 42 facing each other, a second electrochromic layer 60 between the third electrode 32 and the fourth electrode 42, a second electrolyte 70 between the third electrode 32 and the fourth electrode 42 and contacting to the second electrochromic layer 60, and a second ion storage layer 80. It will be understood that in some example embodiments the second ion storage layer 80 may be omitted from the second electrochromic device 100b. It will be understood that in some example embodiments the first ion storage layer 50 may be omitted from the first electrochromic device 100a.

One of the third electrode 32 or the fourth electrode 42 is an operation electrode, and the other may be a counter electrode configured to maintain a flow of electrons. At least one of the third electrode 32 or the fourth electrode 42 may be made into a transparent conductor, which may each include, for example, an inorganic conductive material such as indium tin oxide (ITO), fluorine-doped tin oxide (FTO), antimony-doped tin oxide (ATO), nickel oxide (NiO), titanium oxide (TiO₂) or any combination thereof or an organic conductive material such as polyacetylene, polythiophene, or a derivative thereof.

The second electrochromic layer 60 may include a second electrochromic material differing from the first electrochromic material. The second electrochromic material is the same as described above.

The first electrochromic device 100a and the second electrochromic device 100b may each operate independently. For example, the first electrochromic device 100a and the second electrochromic device 100b may be configured to independently achieve a colored state by oxidation or reduction simultaneously or substantially simultaneously, so that the stacked electrochromic device 200 may display opaque. In another example, the first electrochromic device 100a and the second electrochromic device 100b may be configured to independently achieve a bleached state by oxidation or reduction simultaneously or substantially simultaneously, so that the stacked electrochromic device 200 may display transparent.

For example, the second electrochromic material in the colored state (opaque) may have different light absorption characteristics from those of the first electrochromic material, for example, the second electrochromic material may have a different maximum absorption wavelength (λₘₐₓ₂) in the visible light absorption spectrum from maximum absorption wavelength (λₘₐₓ₁) in the visible light absorption spectrum of the first electrochromic material. For example, the first electrochromic material may be configured to exhibit a first maximum absorption wavelength (λₘₐₓ₁) in the visible light region based on being reversibly converted to a respective colored state of the first electrochromic material to be configured to absorb at least a portion of light in a visible light region, the second electrochromic material may be configured to exhibit a second maximum absorption wavelength (λₘₐₓ₂) in the visible light region based on being reversibly converted to a respective colored state of the second electrochromic material to be configured to absorb at least a portion of light in a visible light region, and the second maximum absorption wavelength of the second electrochromic material may be different from the first maximum absorption wavelength of the first electrochromic material. For example, the maximum absorption wavelength (λₘₐₓ₁) in the visible light absorption spectrum of the first electrochromic material may belong to about 400 nm to 550 nm, and the maximum absorption wavelength (λₘₐₓ₂) in the visible light absorption spectrum of the second electrochromic material may belong to greater than or equal to about 500 nm and less than about 750 nm.

The stacked electrochromic device 200 may display a colored state (opaque) or bleached state (transparent) by combining the first electrochromic device 100a and the second electrochromic device 100b and exhibit light absorption characteristics in the colored (opaque) state, which is a combination of visible light absorption spectra of the first electrochromic material and the second electrochromic material, for example, exhibit the light absorption characteristics over a wavelength region of about 400 nm to 650 nm, for example, display black (e.g., display the color black).

The stacked electrochromic device 200 may reversibly display black or transparent according to the applied voltage. The stacked electrochromic device 200 may be configured to reversibly display black (e.g., absorb all or substantially all visible light) or transparency (e.g., transmit all or substantially all visible light) according to (e.g., based on) the applied voltage. For example, the stacked electrochromic device 200 may be configured to exhibit a first transmittance at a particular wavelength in the visible light region (e.g., a wavelength greater than or equal to about 400 nm to about 700 nm, for example a wavelength of about 470 nm) based on a first voltage being applied thereto (e.g., applied to both the first and second electrochromic layers 30 and 60) and to exhibit a second transmittance at the particular wavelength in the visible light region based on a second voltage being applied thereto (e.g., applied to both the first and second electrochromic layers 30 and 60), where the second voltage is different from the first voltage, and a difference between the first and second transmittances is a difference in light transmittance that the stacked electrochromic device 200 is configured to exhibit at the particular wavelength in the visible light region and may be greater than or equal to about 30% (e.g., about 30% to about 100%). The first and second voltages may be different voltages, including where one of the first and second voltages may be a zero voltage (e.g., 0 V) although example embodiments are not limited thereto, such that the first and second voltages may be different ones of 0 V, 1 V, 1.5 V, 2 V, 2.5 V, or 3 V. Such a difference in light transmittance that the stacked electrochromic device 200 is configured to exhibit at the particular wavelength in the visible light region may be greater than or equal to about 30% in the visible light region (e.g., greater than or equal to about 400 nm and less than or equal to about 700 nm) according to the applied voltage and within the above range, about 30% to about 100%, about 35% to about 100%, about 40% to about 100%, about 45% to about 100%, about 50% to about 100%, about 55% to about 100%, or about 60% to about 100%. Accordingly, the stacked electrochromic device 200 may be configured to exhibit a difference in light transmittance at a wavelength of 470 nm based on a difference in an applied voltage that is applied to the electrochromic device 200, where the difference in light transmittance may be about 30% to about 100%.

The above electrochromic device may be applied to (e.g., included in) various electronic devices that require of controlling an inflow of light, for example, an optical filter, an electron signboard, a smart window, or a wearable imaging device (e.g., an augmented reality (AR) device, a virtual reality (VR) device, or the like.

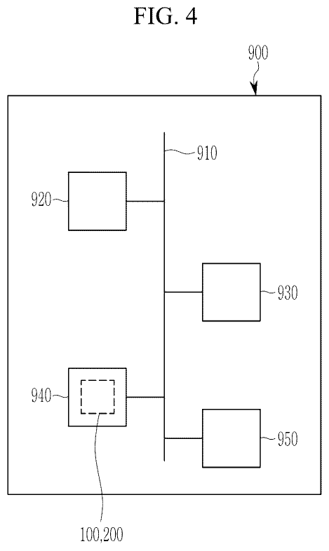

FIG. 4 is a schematic view of an electronic device 900 according to some example embodiments.

Referring to FIG. 4, an electronic device 900 may include a processor 920 (e.g., a central processing unit or CPU), a memory 930 (e.g., a solid-state drive (SSD) storage device), an additional device 940, and a power supply 950 electrically connected to each other through a bus 910. The additional device 940 may include an electrochromic device 100 as described above (e.g., an electrochromic device 100 according to any of the example embodiments), a stacked electrochromic device 200 as described above (e.g., a stacked electrochromic device 200 according to any of the example embodiments), or any combination thereof. For example, the additional device 940 may include an optical filter, an electronic signboard, a smart window, a wearable imaging device and/or display device (such as an augmented reality (AR) or a virtual reality (VR) imaging device and/or display device), any combination thereof, or the like, any of which may include an electrochromic device 100 according to any of the example embodiments. In some example embodiments, the additional device 940 may include a processor (e.g., a CPU) and/or a memory (e.g., an SSD). The memory 930 (and/or a memory of the additional device 940), which may be a non-transitory computer readable medium (e.g., an SSD storage device), may store a program of instructions. The processor 920 (and/or a processor of the additional device 940), for example a CPU, may execute the stored program of instructions to perform one or more functions. The power supply 950 may include a rechargeable battery, such as a lithium-ion battery. The processor 920 (and/or a processor of the additional device 940) may be configured to adjustably control a voltage of electrical power (e.g., electrical power supplied from the power supply 950) applied to one or more elements of an electrochromic device 100 of the additional device 940. For example, the processor 920 (and/or a processor of the additional device 940) may be configured to adjustably control a supply of electrical power from the power supply 950 to the additional device 940, for example to adjustably control a voltage applied to an electrochromic device 100 of the additional device 940, to control a color and/or opacity exhibited by at least an electrochromic layer 30 of the electrochromic device 100. The processor 920 (and/or a processor of the additional device 940) may be configured to generate an output (e.g., cause a certain voltage to be applied to the electrochromic layer 30 of the electrochromic device 100 of the additional device 940, to cause the electrochromic device 100 and/or stacked electrochromic device 200 to exhibit a particular color and/or opacity) based on processing a program of instructions stored at the memory 930 (and/or a memory of the additional device 940). In some example embodiments, the additional device 940 may be electrically connected to the power supply 950 independently of a bus 910, and a processor included in the additional device 940 and/or the power supply 950 may be configured to adjustably control the application of voltage to the electrochromic device 100 and/or stacked electrochromic device 200 based on electrical power supplied from the power supply 950. In some example embodiments, one or more of the bus 910, the processor 920, the memory 930, and/or the power supply 950 may be omitted.

The electronic device 900 and/or any portion thereof (e.g., processor 920, memory 930, additional device 940, power supply 950, etc.) may include processing circuitry such as a hardware including logic circuits; a hardware/software combination such as processor-implemented software; or any combination thereof. For example, the processing circuitry may be a central processing unit (CPU), an arithmetic logic unit (ALU), a digital signal processor, a microcomputer, a field programmable gate array (FPGA), System-on-Chip (SoC), a programmable logic unit, a microprocessor, an application-specific integrated circuit (ASIC), or the like. As an example, the processing circuitry may include a non-transitory computer readable storage device. The processor 920 (e.g., a central processing unit or CPU) may, for example, control an operation of the additional device 940 (e.g., an electrochromic device 100 thereof), for example based on executing an instruction program stored at the memory 930 (e.g., a solid-state drive (SSD) storage device).

Hereinafter, some example embodiments are illustrated in more detail with reference to examples. However, the scope of the inventive concepts is not limited to such examples.

### SYNTHESIS EXAMPLES: SYNTHESIS OF COMPOUND

### Synthesis Example 1

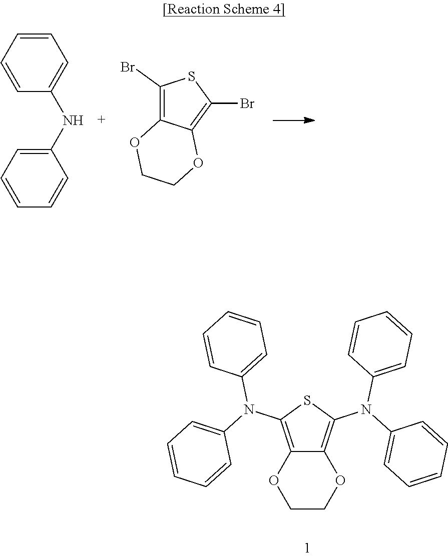 [SMILES: c1ccc(Nc2ccccc2)cc1 ; Brc1sc(Br)c2c1OCCO2 ; c1ccc(N(c2ccccc2)c2sc(N(c3ccccc3)c3ccccc3)c3c2OCCO3)cc1]

3.10 g of diphenylamine and 2.50 g of 2,5-dibromo-3,4-ethylenedioxythiophene are heated with 1.53 g of tris(dibenzylideneacetone)dipalladium (0) (Pd₂(dba)₃), 1.35 ml of a 50% tri-t-butylphosphine (P(t-Bu)₃) toluene solution, and 3.20 g of sodium t-butoxide (NaOtBu) in 150 ml of toluene at 100° C. for 12 hours. The obtained product is separated and purified through silica gel column chromatography (ethyl acetate:hexane=1:9 v/v) to obtain 1.60 g (a yield of 40%) of Compound 1.

1H-NMR (500 MHz, Methylene Chloride-d2): δ 7.29 (t, 8H), 7.11 (d, 8H), 7.03 (t, 4H), 4.17 (s, 4H).

### Synthesis Example 2

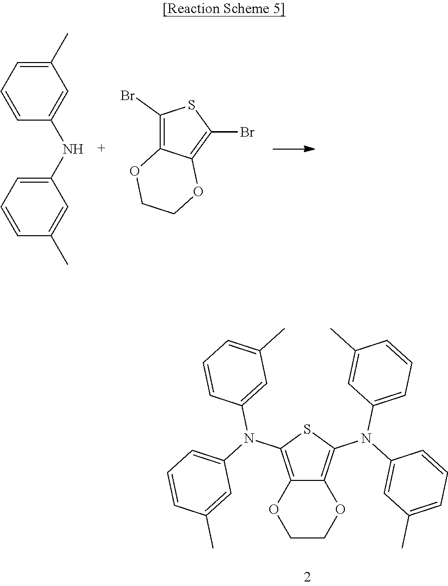 [SMILES: Cc1cccc(Nc2cccc(C)c2)c1 ; Brc1sc(Br)c2c1OCCO2 ; Cc1cccc(N(c2cccc(C)c2)c2sc(N(c3cccc(C)c3)c3cccc(C)c3)c3c2OCCO3)c1]

3.62 g of m,m′-ditolylamine and 2.50 g of 2,5-dibromo-3,4-ethylenedioxythiophene are heated with 1.53 g of Pd₂(dba)₃, 1.4 ml of a 50% P(t-Bu)₃ toluene solution, and 3.2 g of NaOtBu in 150 ml of toluene at 100° C. for 12 hours. The obtained product is separated and purified through silica gel column chromatography (ethyl acetate:hexane=1:9 v/v) to obtain 2.0 g of Compound 2. The yield is about 45%.

1H NMR (500 MHz, Methylene Chloride-d2) δ 7.17 (t, 4H), 6.94-6.82 (m, 12H), 4.18 (s, 4H), 2.30 (s, 12H)

### MANUFACTURING EXAMPLES I: MANUFACTURE OF ELECTROCHROMIC DEVICES Example 1

A glass plate coated with ITO (Indium Tin Oxide) is washed in water, acetone, and ethanol solvents by using an ultrasonic wave and then, dried at 70° C. in an oven to prepare an ITO glass plate. On the ITO glass plate, a NiO nanoparticle solution (avantama P-21, Avantama AG, a distribution of diameters of the NiO nanoparticles: about 5-50 nm) is spin-coated at a rotation speed of 1000 rpm for 30 seconds and then, dried to form a 250 nm-thick NiO layer (ion storage layer). Subsequently, the ITO glass plate coated with the NiO layer is disposed to face a bare ITO glass plate, and a space for a solution is defined therebetween by using polydimethyl siloxane (PDMS) (0.5 mm).

Subsequently, the solution obtained by dissolving Compound 1 of Synthesis Example 1 (2 mM, an electrochromic material) and LiClO₄ (0.1 M, an electrolyte) in a mixed solvent of acetonitrile and toluene (in a ratio of 1:2 v/v) is poured between the ITO glass plate coated with the NiO layer and the bare ITO glass plate, which are closed and sealed to manufacture an electrochromic device.

### Example 2

An electrochromic device is manufactured in the same manner as in Example 1 except that Compound 2 of Synthesis Example 2 is used instead of Compound 1 of Synthesis Example 1.

### Comparative Example 1

After preparing two sheets of ITO glass plates, an NiO nanoparticle solution is spin-coated on each of the ITO glass plates at a rotation speed of 1000 rpm for 30 seconds and then, dried to form a 250 nm-thick NiO layer. The two ITO glass plates coated with the NiO layers are disposed to face each other, and a space for an electrolyte solution is defined by using polydimethylsiloxane (PDMS) (0.5 mm). Subsequently, the electrolyte solution prepared by dissolving LiClO₄ (0.1 M) in a mixed solvent of acetonitrile and toluene (in a ratio of 1:2 v/v) is poured into between the ITO glass plates coated with the NiO layers, which are closed and sealed to manufacture an electrochromic device. Herein, one of the NiO layers may function as an electrochromic layer, and the other one of the NiO layers may function as an ion storage layer.

### Comparative Example 2

A NiO nanoparticle solution is spin-coated on an ITO glass plate at a rotation speed of 1000 rpm for 30 seconds and then, dried to form a 250 nm-thick NiO layer (ion storage layer). The ITO glass plate coated with the NiO layer is disposed to face a bare ITO glass plate, and a space for an electrolyte solution is defined therebetween by using polydimethylsiloxane (PDMS) (0.5 mm). Subsequently, the electrolyte solution is prepared by dissolving aniline (2 mM) and H₂SO₄ (0.5 M) in water and then, poured into the space between the ITO glass plate coated with the NiO layer and the bare ITO glass plate, which are closed and sealed.

Then, the ITO glass plate is coated with polyaniline (PANI) by an electro-polymerization of 5 cycles applying a voltage of 0 V to 2 V to ITO. Subsequently, the ITO glass plate coated with the polyaniline is separated.

After disposing the ITO glass plate coated with polyaniline to face the ITO glass plate coated with NiO, an electrolyte solution prepared by dissolving LiClO₄ (0.1 M) in a mixed solvent of acetonitrile and toluene (in a ratio of 1:2 v/v) is poured between the ITO glass plate coated with polyaniline and the ITO glass plate coated with NiO, which are closed and sealed to manufacture an electrochromic device.

### Evaluation I

The electrochromic devices according to Examples 1 and 2 and Comparative Examples 1 and 2 are evaluated with respect to electrochromic properties.

A potentiostat (Compactstat.h, Ivium Technologies B.V.) is used to apply a voltage to the electrochromic devices, and a Lambda 750 UV-Vis spectrophotometer (PerkinElmer Inc.) is used to measure absorbance and light transmittance, which are output by electrochromic properties of the electrochromic devices.

A constant current is input to the electrochromic devices according to Examples 1 and 2 and Comparative Examples 1 and 2 by Chronoamperometry, and simultaneously, absorbance and transmittance output therefrom are monitored in real time by using UV-Vis spectroscopy.

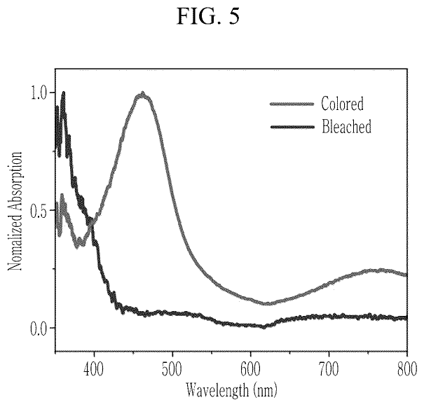

The results are shown in FIGS. 5 to 8.

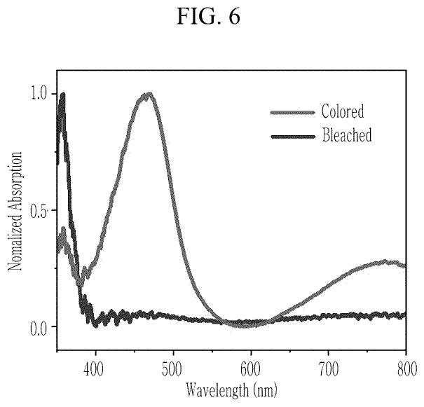

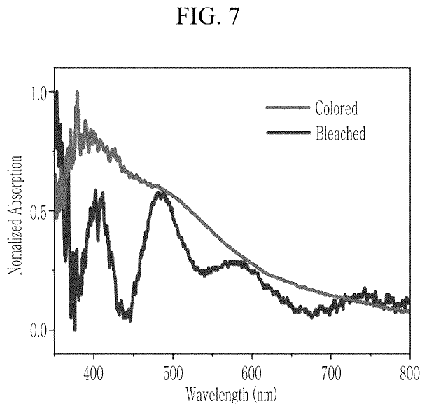

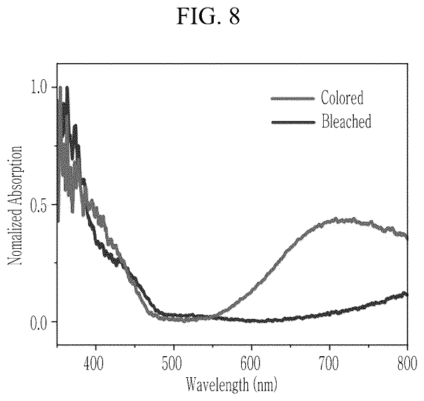

FIG. 5 is a UV-Vis absorption spectrum of the electrochromic device according to Example 1 in a colored state and a bleached state, FIG. 6 is a UV-Vis absorption spectrum of an electrochromic device according to Example 2 in a colored state and a bleached state, FIG. 7 is a UV-Vis absorption spectrum of the electrochromic device according to Comparative Example 1 in a colored state and a bleached state, and FIG. 8 is a UV-Vis absorption spectrum of the electrochromic device according to Comparative Example 2 in a colored state and a bleached state.

Referring to FIGS. 5 and 6, the electrochromic devices according to Examples 1 and 2 have virtually no substantial light absorption in the visible light region at a voltage of 0 V (bleached state, “Bleached”) but when applying a voltage of 2 V thereto, exhibit high light absorption in the visible light region (maximum absorption wavelength: about 470 nm) (colored state, “Colored”). Accordingly, it is confirmed that the electrochromic devices according to Examples 1 and 2 may exhibit a large color contrast according to the applied voltage.

On the contrary, referring to FIGS. 7 and 8, the electrochromic devices according to Comparative Examples 1 and 2, to which voltages of 0 V and 2 V are applied, exhibit light absorption at visible light wavelengths, which may confirm a small color contrast of bleached state (“Bleached”) and colored state (“Colored”), and particularly, the electrochromic device according to Comparative Example 2 exhibits almost no light absorption characteristic difference of the bleached state (“Bleached”) and the colored state (“Colored”), which confirms no substantial color contrast.

### MANUFACTURING EXAMPLES II: MANUFACTURE OF ELECTROCHROMIC DEVICES

### Example 3

An NiO nanoparticle solution is spin-coated on an ITO glass plate at a rotation speed of 1000 rpm for 30 seconds and then, dried to form a 250 nm-thick NiO layer (ion storage layer). Subsequently, the ITO glass plate coated with the NiO layer is disposed to face a bare ITO glass plate, and a space for a solution is defined by using polydimethylsiloxane (PDMS) (0.5 mm).

The solution is prepared by dissolving 3,4-(2,2-dimethylpropylenedioxy)thiophene (2 mM) and LiClO₄ (0.5 M) in a mixed solvent (in a ratio of 1:2 v/v) of acetonitrile and toluene and poured into the space between the ITO glass plate coated with the NiO layer and the bare ITO glass plate, which are closed and sealed.

Subsequently, the ITO glass plate is coated with poly-3,4-(2,2-dimethylpropylenedioxy)thiophene (a thickness of 300 nm, a first electrochromic material) by an electro-polymerization of 5 cycles applying a voltage of 0 V to 3 V to ITO. Subsequently, the ITO glass plate coated with the poly-3,4-(2,2-dimethylpropylenedioxy)thiophene is separated.

Subsequently, the ITO glass plate coated with the poly-3,4-(2,2-dimethylpropylenedioxy)thiophene is disposed to face a bare ITO glass plate, and a space for a solution is defined therebetween by using polydimethylsiloxane (PDMS) (0.5 mm).

Subsequently, the solution is prepared by dissolving Compound 1 of Synthesis Example 1 (2 mM, a second electrochromic material) and LiClO₄ (0.1 M, an electrolyte) in a mixed solvent of acetonitrile and toluene (in a ratio of 1:2 v/v) and then, poured into the space between the ITO glass plate coated with the poly-3,4-(2,2-dimethylpropylenedioxy)thiophene and the bare ITO glass plate, which are closed and sealed to manufacture an electrochromic device.

### Evaluation II

The electrochromic device according to Example 3 is evaluated with respect to electrochromic properties.

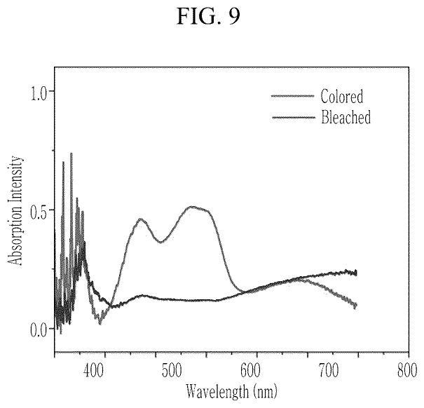

The results are shown in FIG. 9.

FIG. 9 is a UV-Vis absorption spectrum of the electrochromic device according to Example 3 in a colored state and a bleached state.

Referring to FIG. 9, the electrochromic device according to Example 3 had no substantial light absorption in the visible light region (bleached state, “Bleached”) when a voltage of −3 V is applied thereto, but when a voltage of 3 V is applied thereto, exhibits high light absorption in a relatively large visible light region (about 400 nm to 600 nm) (colored state, “Colored”). Accordingly, it is confirmed that the electrochromic device according to Example 3 may effectively display reversibly black or transparency according to the applied voltages.

### MANUFACTURING EXAMPLES III: MANUFACTURE OF ELECTROCHROMIC DEVICES

### Example 4

### (1) Manufacturing of First Electrochromic Device

An NiO nanoparticle solution is spin-coated on an ITO glass plate at a rotation speed of 1000 rpm for 30 seconds and then, dried to form a 250 nm-thick NiO layer (a first ion storage layer). Subsequently, the ITO glass plate coated with the NiO layer is disposed to face a bare ITO glass plate, and polydimethylsiloxane (PDMS) (0.5 mm) is disposed therebetween.

Subsequently, a solution is prepared by dissolving Compound 1 of Synthesis Example 1 (2 mM, a first electrochromic material) and LiClO₄ (0.1 M and an electrolyte) in a mixed solvent of acetonitrile and toluene (in a ratio of 1:2 v/v) and then, poured into between the ITO glass plate coated with the NiO layer and the bare ITO glass plate, which are closed and sealed to manufacture a first electrochromic device.

### (2) Manufacturing of Second Electrochromic Device

An NiO nanoparticle solution is spin-coated on an ITO glass plate at a rotation speed of 1000 rpm for 30 seconds and then, dried to form a 250 nm-thick NiO layer (a second ion storage layer). Subsequently, the ITO glass plate coated with the NiO layer is disposed to face a bare ITO glass plate, and a space for a solution is defined by using polydimethylsiloxane (PDMS) (0.5 mm).

The solution is prepared by dissolving 3,4-(2,2-dimethylpropylenedioxy)thiophene (2 mM) and LiClO₄ (0.5 M) in a mixed solvent of acetonitrile and toluene (in a ratio of 1:2 v/v) and then, poured into the space between the ITO glass plate coated with the NiO layer and the bare ITO glass plate, which are closed and sealed.

Subsequently, the ITO glass plate is coated with the poly-3,4-(2,2-dimethylpropylenedioxy)thiophene (a thickness of 300 nm, a second electrochromic material) by an electro-polymerization of 5 cycles applying a voltage of 0 V to 3 V to ITO. Subsequently, the ITO glass plate coated with the poly-3,4-(2,2-dimethylpropylenedioxy)thiophene is separated.

An ITO glass plate coated with a NiO layer is prepared in the same method as above. Subsequently, the ITO glass plate coated with the poly-3,4-(2,2-dimethylpropylenedioxy)thiophene and the ITO glass plate coated with the NiO layer are disposed to face each other, and an electrolyte solution prepared by dissolving LiClO₄ (0.1 M) in a mixed solvent (in a ratio of 1:2 v/v) of acetonitrile and toluene is poured between the ITO glass plate coated with the poly-3,4-(2,2-dimethylpropylenedioxy)thiophene and the ITO glass plate coated with the NiO layer, which are closed and sealed to manufacture a second electrochromic device.

### (3) Manufacturing of Stacked Electrochromic Device

A stacked electrochromic device is manufactured by disposing and assembling the first electrochromic device and the second electrochromic device so as to overlap each other.

### Evaluation III

The stacked electrochromic device according to Example 4 is evaluated with respect to electrochromic properties.

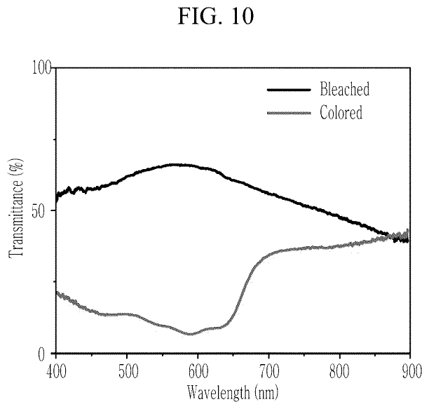

The results are shown in FIG. 10.

FIG. 10 shows transmission spectra of the electrochromic device according to Example 4 in the colored and bleached states.

Referring to FIG. 10, the stacked electrochromic device according to Example 4, in which a voltage of 0 V is applied to the first electrochromic device, while a voltage of 1.5 V is applied to the second electrochromic device, exhibits high transmittance in the visible light region (bleached state, “Bleached”), but in which a voltage of 0 V is applied to the second electrochromic device, while a voltage of 2.5 V is applied to the first electrochromic device, exhibits low transmittance in the visible light region (colored state, “Colored”). Accordingly, it is confirmed that the stacked electrochromic device according to Example 4 may effectively display reversibly black or transparence (“transparency”) according to the applied voltages.

Hereabove, some example embodiments have been described in detail, but the scope of the inventive concepts is not limited thereto but also includes various modifications and improvements made by those skilled in the art by using the basic concepts defined in the following claims.

What is claimed is:

1. A compound represented by Chemical Formula 1: 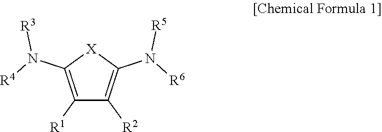
wherein, in Chemical Formula 1,
X is O, S, Se, or Te,
R¹ and R² are each independently hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
at least one of R¹ or R² is a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
R¹ and R² are each independently present or are linked to each other to form a ring,
R³ to R⁶ are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
at least one of R³ or R⁴ is a substituted or unsubstituted C6 to C30 aryl group, and
at least one of R⁵ or R⁶ is a substituted or unsubstituted C6 to C30 aryl group.

2. The compound of claim 1, wherein, in Chemical Formula 1,
at least one of R³ or R⁴ is a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group, and
at least one of R⁵ or R⁶ is a substituted or unsubstituted phenyl group, a substituted or unsubstituted biphenyl group, a substituted or unsubstituted naphthyl group, a substituted or unsubstituted terphenyl group, a substituted or unsubstituted anthracenyl group, a substituted or unsubstituted fluorenyl group, or a substituted or unsubstituted triphenylenyl group.

3. The compound of claim 1, wherein, in Chemical Formula 1,
R¹ and R² are each independently a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, or any combination thereof, and
R¹ and R² are each independently present or linked to each other to form a ring.

4. The compound of claim 1, wherein the compound is represented by Chemical Formula 1A: 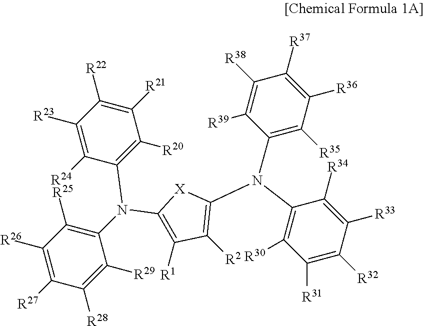
wherein, in Chemical Formula 1A,
X is O, S, Se, or Te,
R¹ and R² are each independently hydrogen, a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
at least one of R¹ or R² is a substituted or unsubstituted C1 to C30 alkyl group, a substituted or unsubstituted C1 to C30 alkoxy group, a substituted or unsubstituted C1 to C30 alkylthio group, a substituted or unsubstituted C1 to C30 alkylseleno group, a substituted or unsubstituted C1 to C30 alkyltelluro group, a hydroxyl group, a thiol group, a substituted or unsubstituted C6 to C30 aryl group, a substituted or unsubstituted C3 to C30 heteroaryl group, or any combination thereof,
R¹ and R² are each independently present or are linked to each other to form a ring,
R²⁰ to R³⁹ are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C6 to C30 aryl group, a substituted or unsubstituted C3 to C30 heteroaryl group, a hydroxyl group, a halogen, or any combination thereof, and
R²⁰ to R³⁹ are each independently present or two adjacent ones of R²⁰ to R³⁹ are linked to each other to form a ring.

5. The compound of claim 1, wherein the compound is represented by Chemical Formula 1B-1 or Chemical Formula 1B-2: 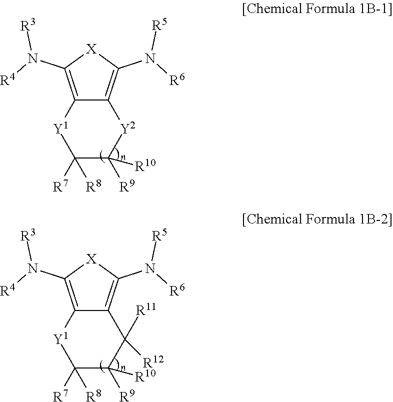
wherein, in Chemical Formula 1B-1 and Chemical Formula 1B-2,
X, Y¹, and Y² are each independently O, S, Se, or Te,
R³ to R¹² are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
R⁷ to R¹² are each independently present or two adjacent ones of R⁷ to R¹² are linked to each other to form a ring,
at least one of R³ or R⁴ is a substituted or unsubstituted C6 to C30 aryl group,
at least one of R⁵ or R⁶ is a substituted or unsubstituted C6 to C30 aryl group, and
n is an integer of 0 to 3.

6. The compound of claim 1, wherein the compound is represented by Chemical Formula 1C-1 or Chemical Formula 1C-2: 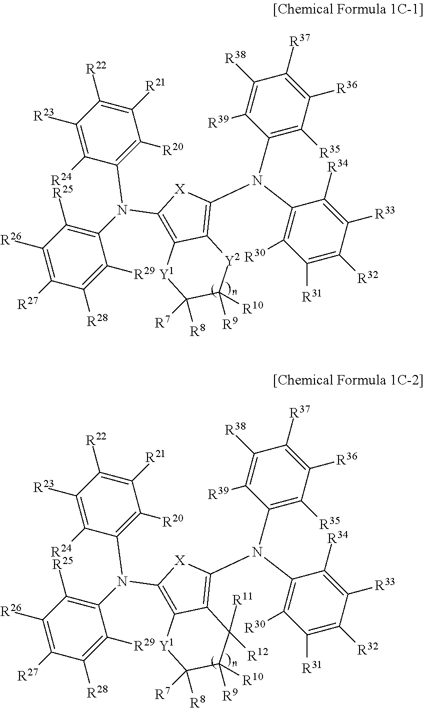
wherein, in Chemical Formula 1C-1 and Chemical Formula 1C-2,
X, Y¹, and Y² are each independently O, S, Se, or Te,
R⁷ to R¹² and R²⁰ to R³⁹ are each independently hydrogen, a substituted or unsubstituted C1 to C30 alkyl group, a substituted or unsubstituted C2 to C30 alkenyl group, a substituted or unsubstituted C2 to C30 alkynyl group, a substituted or unsubstituted C1 to C30 alkoxy group, a substituted or unsubstituted C1 to C30 alkylthio group, a substituted or unsubstituted C6 to C30 aryl group, a substituted or unsubstituted C3 to C30 heteroaryl group, a hydroxyl group, a halogen, or any combination thereof,
R⁷ to R¹² are each independently present or two adjacent ones of R⁷ to R¹² are linked to each other to form a ring,
R²⁰ to R³⁹ are each independently present or two adjacent ones of R²⁰ to R³⁹ are linked to each other to form a ring, and
n is an integer of 0 to 3.

7. The compound of claim 1, wherein the compound is an electrochromic material that is configured to be reversibly converted, based on an applied voltage, into
a colored state wherein the electrochromic material is configured to absorb at least a portion of light in a visible light region to display a color, or
a bleached state wherein the electrochromic material is configured to transmit light in the visible light region, the transmitted light including the portion of light in the visible light region.

8. A polymer comprising a structural unit derived from the compound of claim 1.

9. An electrochromic device, comprising:
a first electrode and a second electrode;
a first electrochromic layer between the first electrode and the second electrode, the first electrochromic layer including a first electrochromic material; and
a first electrolyte in contact with the first electrochromic layer,
wherein the first electrochromic material includes the compound of claim 1, a polymer including a structural unit derived from the compound, or any combination thereof.

10. The electrochromic device of claim 9, wherein the polymer is a reaction product of electro-polymerization using at least one of R³ to R⁶ of Chemical Formula 1 as a reaction site.

11. The electrochromic device of claim 9, wherein the electrochromic device is configured to exhibit a difference in light transmittance at a wavelength of 470 nm based on a difference in an applied voltage that is applied to the electrochromic device, the difference in light transmittance about 30% to about 100%.

12. The electrochromic device of claim 9, further comprising:
a second electrochromic layer between the first electrode and the second electrode,
wherein the second electrochromic layer comprises a second electrochromic material, the second electrochromic material different from the first electrochromic material, and
the second electrochromic material is configured to be reversibly converted, based on an applied voltage, into
a colored state wherein the second electrochromic material is configured to absorb at least a portion of light in a visible light region to display a color, or
a bleached state wherein the second electrochromic material is configured to transmit light in the visible light region, the transmitted light including the portion of light in the visible light region.

13. The electrochromic device of claim 12, wherein
the first electrochromic material is configured to exhibit a first maximum absorption wavelength in the visible light region based on being reversibly converted to a colored state of the first electrochromic material to be configured to absorb at least a portion of light in the visible light region,
the second electrochromic material is configured to exhibit a second maximum absorption wavelength in the visible light region based on being reversibly converted to the colored state of the second electrochromic material, and
the second maximum absorption wavelength of the second electrochromic material is different from the first maximum absorption wavelength of the first electrochromic material.

14. The electrochromic device of claim 12, wherein
the electrochromic device is configured to reversibly display black or transparency based on the applied voltage, and
the electrochromic device is configured to exhibit a difference in light transmittance in the visible light region based on a difference in the applied voltage, wherein the difference in light transmittance in the visible light region is about 30% to about 100%.

15. A stacked electrochromic device, comprising:
a first electrochromic device, the first electrochromic device including the electrochromic device of claim 9; and
a second electrochromic device, the second electrochromic device stacked with the first electrochromic device,
wherein the second electrochromic device includes
a third electrode and a fourth electrode,
a second electrochromic layer between the third electrode and the fourth electrode, and
a second electrolyte in contact with the second electrochromic layer.

16. The stacked electrochromic device of claim 15, wherein
the second electrochromic layer comprises a second electrochromic material, the second electrochromic material different from the first electrochromic material, and
the second electrochromic material is configured to, based on an applied voltage, be reversibly converted into
a colored state wherein the second electrochromic material is configured to absorb at least a portion of light in a visible light region to display a color, or
a bleached state wherein the second electrochromic material is configured to transmit light in the visible light region, the transmitted light including the portion of light in the visible light region.

17. The stacked electrochromic device of claim 16, wherein
the first electrochromic material is configured to exhibit a first maximum absorption wavelength in the visible light region based on being reversibly converted to a colored state of the first electrochromic material to be configured to absorb at least a portion of light in the visible light region,
the second electrochromic material is configured to exhibit a second maximum absorption wavelength in the visible light region based on being reversibly converted to the colored state of the second electrochromic material, and
the second maximum absorption wavelength of the second electrochromic material is different from the first maximum absorption wavelength of the first electrochromic material.

18. The stacked electrochromic device of claim 16, wherein
The stacked electrochromic device is configured to reversibly display black or transparency based on the applied voltage, and
the electrochromic device is configured to exhibit a difference in light transmittance in the visible light region based on a difference in the applied voltage, wherein the difference in light transmittance in the visible light region is about 30% to about 100%.

19. An electronic device comprising the electrochromic device of claim 9.

20. An electronic device comprising the stacked electrochromic device of claim 15.
